(19) United States
(12) Patent Application Publication
(10) Pub. No.: US 2014/0057908 A1
(43) Pub. Date: Feb. 27, 2014

(54) Method of Treating Cancer

(71) Applicant: Exelixis, Inc., South San Francisco, CA (US)

(72) Inventors: David C. Smith, Ann Arbor, MI (US); Maha Hussain, Ann Arbor, MI (US)

(73) Assignee: Exelixis, Inc., South San Francisco, CA (US)

(21) Appl. No.: 13/876,306

(22) PCT Filed: Sep. 26, 2011
(86) PCT No.: PCT/US11/53245

§ 371 Date: Nov. 8, 2013

Related U.S. Application Data
(60) Provisional application No. 61/386,971, filed on Sep. 27, 2010.
(60) Provisional application No. 61/386,983, filed on Sep. 27, 2010.
(60) Provisional application No. 61/386,993, filed on Sep. 27, 2010.

(51) Int. Cl. C07D 215/22

(52) U.S. Cl. CPC C07D 215/22
USPC 514/235.2

(57) ABSTRACT

This invention is directed to the treatment of cancer, particularly castration-resistant prostate cancer and osteoblastic bone metastases, with a dual inhibitor of MET and VEGF.

### CROSS-REFERENCE TO RELATED APPLICATIONS

This application claims the benefit of priority to U.S. Provisional Application No. 61/386,971, filed Sep. 27, 2010, 61/386,993, filed Sep. 27, 2010, and 61/386,983, filed Sep. 27, 2010, which is incorporated herein by reference.

### FIELD OF THE INVENTION

This invention is directed to the treatment of cancer, particularly castration-resistant prostate cancer and osteoblastic bone metastases, with a dual inhibitor of MET and VEGF.

### BACKGROUND OF THE INVENTION

Castration-Resistant Prostate Cancer (CRPC) is a leading cause of cancer-related death in men. Despite progress in systemic therapy for CRPC, improvements in survival are modest, and virtually all patients succumb to this disease with a median survival of about 2 years. The primary cause of morbidity and mortality in CRPC is metastasis to the bone, which occurs in about 90% of cases.

Metastasis to bone is a complex process involving interactions between cancer cells and components of the bone microenvironment including osteoblasts, osteoclasts, and endothelial cells. Bone metastases cause local disruption of normal bone remodeling, and lesions generally show a propensity for either osteoblastic (bone-forming) or osteolytic (bone-resorbing) activity. Although most CRPC patients with bone metastases display features of both types of lesions, prostate cancer bone metastases are often osteoblastic, with abnormal deposition of unstructured bone accompanied by increased skeletal fractures, spinal cord compression, and severe bone pain.

The receptor tyrosine kinase MET plays important roles in cell motility, proliferation, and survival, and has been shown to be a key factor in tumor angiogenesis, invasiveness, and metastasis. Prominent expression of MET has been observed in primary and metastatic prostate carcinomas, with evidence for higher levels of expression in bone metastases compared to lymph node metastases or primary tumors.

Vascular endothelial growth factor (VEGF) and its receptors on endothelial cells are widely accepted as key mediators in the process of tumor angiogenesis. In prostate cancer, elevated VEGF in either plasma or urine is associated with shorter overall survival. VEGF may also play a role in activating the MET pathway in tumor cells by binding to neuropilin-1, which is frequently up-regulated in prostate cancer and appears to activate MET in a co-receptor complex. Agents targeting the VEGF signaling pathway have demonstrated some activity in patients with CRPC.

Thus, a need remains for methods of treating prostate cancer including CRPC and the associated osteoblastic bone metastases.

### SUMMARY OF THE INVENTION

These and other needs are met by the present invention which is directed to a method for treating bone cancer, prostate cancer, or bone cancer associated with prostate cancer. The method comprises administering a therapeutically effective amount of a compound that modulates both MET and VEGF to a patient in need of such treatment. In one embodiment, the bone cancer is osteoblastic bone metastases. In a further embodiment, the prostate cancer is CRPC. In a further embodiment, the bone cancer is osteoblastic bone metastases associated with CRPC.

In one aspect, the present invention is directed to a method for treating osteoblastic bone metastases, CRPC, or osteoblastic bone metastases associated with CRPC, comprising administering a therapeutically effective amount of a compound that dually modulates MET and VEGF to a patient in need of such treatment.

In one embodiment of this and other aspects, the dual acting METNEGF inhibitor is a compound of Formula I as provided in Exhibit A.

In one embodiment of this and other aspects, the dual acting MET/VEGF inhibitor is a compound of Formula I:

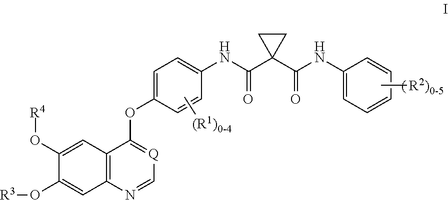

or a pharmaceutically acceptable salt thereof, wherein:

R¹ is halo;

R² is halo;

R³ is (C₁-C₆)alkyl or (C₁-C₆)alkyl optionally substituted with heterocycloalkyl;

R⁴ is (C₁-C₆)alkyl; and

Q is CH or N.

In another embodiment, the compound of Formula I is Compound 1:

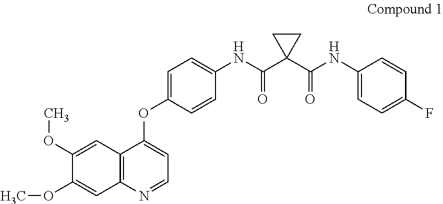 [SMILES: COc1cc2nccc(Oc3ccc(NC(=O)C4(C(=O)Nc5ccc(F)cc5)CC4)cc3)c2cc1OC]

or a pharmaceutically acceptable salt thereof. Compound 1 is known as N-(4-{[6,7-bis(methyloxy)quinolin-4-yl]oxy}phenyl)-N′-(4-fluorophenyl)cyclopropane-1,1-dicarboxamide. WO 2005/030140 describes the synthesis of N-(4-{[6,7-bis(methyloxy)quinolin-4-yl]oxy}phenyl)-N′-(4-fluorophenyl)cyclopropane-1,1-dicarboxamide (Example 12, 37, 38, and 48) and also discloses the therapeutic activity of this molecule to inhibit, regulate and/or modulate the signal transduction of kinases, (Assays, Table 4, entry 289). Example 48 is on paragraph [0353] in WO 2005/030140.

In another embodiment, the compound of Formula I is Compound 2:

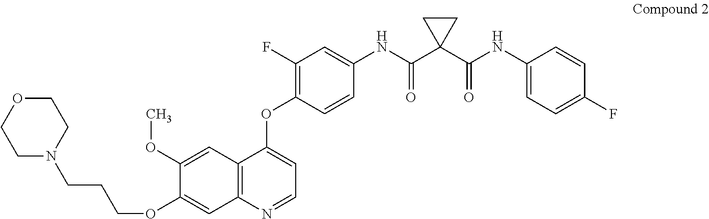 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt thereof. Compound 2 is known as is N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide. WO 2005-030140 describes the synthesis of Compound (I) (Examples 25, 30, 36, 42, 43 and 44) and also discloses the therapeutic activity of this molecule to inhibit, regulate and/or modulate the signal transduction of kinases, (Assays, Table 4, entry 312). Compound 2 has been measured to have a c-Met IC₅₀ value of about 0.6 nanomolar (nM). PCT/US09/064,341, which claims priority to U.S. provisional application 61/199,088, filed Nov. 13, 2008, describes a scaled-up synthesis of Compound I.

In another embodiment, the invention provides a method of a method for treating osteoblastic bone metastases associated with CRPC, comprising administering a therapeutically effective amount of a pharmaceutical formulation comprising Compound of Formula I or the malate salt of Compound of Formula I or another pharmaceutically acceptable salt of Compound of Formula I, to a patient in need of such treatment.

In another embodiment, the dual MET/VEGF inhibitor is a compound of Formula II:

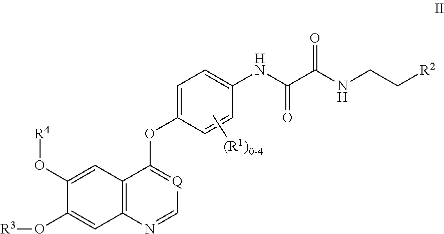

or a pharmaceutically acceptable salt thereof, wherein:

R¹ is halo;

R² is optionally substituted phenyl;

R³ is (C₁-C₆)alkyl substituted with heterocycloalkyl;

R⁴ is (C₁-C₆)alkyl; and

Q is CH or N.

In another embodiment, the compound of Formula II is Compound 3:

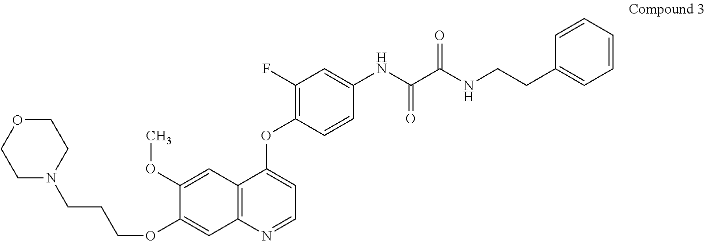 [SMILES: COc1cc2c(Oc3ccc(NC(=O)C(=O)NCCc4ccccc4)cc3F)ccnc2cc1OCCCN1CCOCC1]

or a pharmaceutically acceptable salt thereof. Compound 3 is disclosed in WO 2005-030140, which describes the synthesis of Compound 3 and also discloses the therapeutic activity of this molecule to inhibit, regulate and/or modulate the signal transduction of kinases. Compound 3 is specifically disclosed in Table 1 of WO 2005-030140 as Example 41, pages 206-207. The biological activity for Compound 1 is disclosed in Table 4 as compound 137 on page 275.

In another embodiment, the invention provides a method for treating bone cancer, prostate cancer, or bone cancer associated with prostate cancer, comprising administering a composition comprising:

(a) one or more inhibitor(s) of VEGFR; and

(b) one or more inhibitor(s) of MET

to a patient in need of such treatment.

In certain embodiments, the prostate cancer is CRPC. In other embodiments, the bone cancer is osteoblastic bone metastasis.

### BRIEF DESCRIPTION OF THE FIGURES

FIGS. 1A-C show the bone scan (FIG. 1A), bone scan response (FIG. 1B), and CT scan data (FIG. 1C) for Patient 1.

FIGS. 2A-C show the bone scan (FIG. 2A), bone scan response (FIG. 2B), and CT scan data (FIG. 2C) for Patient 2.

FIGS. 3A-B show the bone scan (FIG. 3A), bone scan response (FIG. 3B) for Patient 3.

### DETAILED DESCRIPTION OF THE INVENTION

### Abbreviations and Definitions

The following abbreviations and terms have the indicated meanings throughout:

| Abbreviation | Meaning |
|---|---|
| Ac | Acetyl |
| Br | Broad |
| ° C. | degrees Celsius |
| c- | Cyclo |
| CBZ | CarboBenZoxy = benzyloxycarbonyl |
| d | Doublet |
| dd | doublet of doublet |
| dt | doublet of triplet |
| DCM | Dichloromethane |
| DME | 1,2-dimethoxyethane |
| DMF | N,N-dimethylformamide |
| DMSO | dimethyl sulfoxide |
| Dppf | 1,1′-bis(diphenylphosphano)ferrocene |
| EI | Electron Impact ionization |
| G | gram(s) |
| h or hr | hour(s) |
| HPLC | high pressure liquid chromatography |
| L | liter(s) |
| M | molar or molarity |
| m | Multiplet |
| Mg | milligram(s) |
| MHz | megahertz (frequency) |
| Min | minute(s) |
| mL | milliliter(s) |
| μL | microliter(s) |
| μM | Micromole(s) or micromolar |
| mM | Millimolar |
| Mmol | millimole(s) |
| Mol | mole(s) |
| MS | mass spectral analysis |
| N | normal or normality |
| nM | Nanomolar |
| NMR | nuclear magnetic resonance spectroscopy |
| q | Quartet |
| RT | Room temperature |
| s | Singlet |
| t or tr | Triplet |
| TFA | trifluoroacetic acid |
| THF | Tetrahydrofuran |
| TLC | thin layer chromatography |

The symbol “—” means a single bond, “═” means a double bond.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

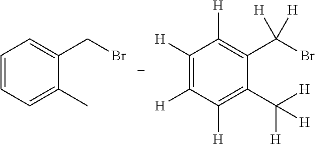

If a group “R” is depicted as “floating” on a ring system, as for example in the formula:

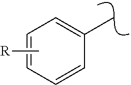

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

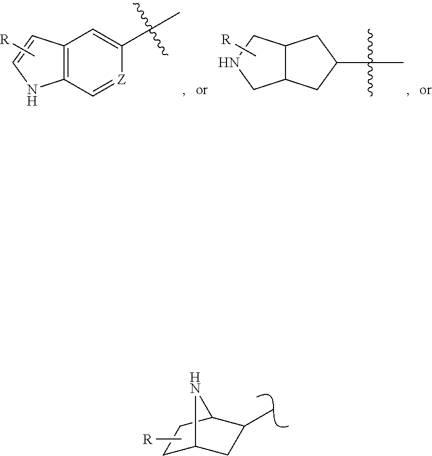

then, unless otherwise defined, a substituent “R” may reside on any atom of the fused ring system, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals ═CH—) from one of the ring atoms, so long as a stable structure is formed. In the example depicted, the “R” group may reside on either the 5-membered or the 6-membered ring of the fused ring system. In the formula depicted above, when y is 2 for example, then the two “R's” may reside on any two atoms of the ring system, again assuming each replaces a depicted, implied, or expressly defined hydrogen on the ring.

When a group “R” is depicted as existing on a ring system containing saturated carbons, as for example in the formula:

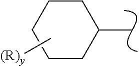

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

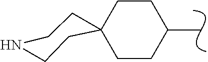

“Halogen” or “halo” refers to fluorine, chlorine, bromine or iodine.

“Yield” for each of the reactions described herein is expressed as a percentage of the theoretical yield.

“Patient” for the purposes of the present invention includes humans and other animals, particularly mammals, and other organisms. Thus the methods are applicable to both human therapy and veterinary applications. In another embodiment the patient is a mammal, and in another embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, malic acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Therapeutically effective amount” is an amount of a compound of the invention, that when administered to a patient, ameliorates a symptom of the disease. A therapeutically effective amount is intended to include an amount of a compound alone or in combination with other active ingredients effective to modulate c-Met, and/or VEGFR, or effective to treat or prevent cancer. The amount of a compound of the invention which constitutes a “therapeutically effective amount” will vary depending on the compound, the disease state and its severity, the age of the patient to be treated, and the like. The therapeutically effective amount can be determined by one of ordinary skill in the art having regard to their knowledge and to this disclosure.

“Treating” or “treatment” of a disease, disorder, or syndrome, as used herein, includes (i) preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experience.

It should be appreciated that methods of the invention may be applicable to various species of subjects, preferably mammals, more preferably humans.

As used herein, the compounds of the present invention include the pharmaceutically acceptable derivatives thereof.

Where the plural form is used for compounds, salts, and the like, this is taken to mean also a single compound, salt and the like.

The terms “combination” and “cotherapy” are used interchangeably herein. The terms “combination” and “cotherapy” refer herein to the administration of a single formulation comprising at least two active agents, as well as sequential administration of at least two active agents or formulations thereof.

The terms “cancer” and “cancerous” when used herein refer to or describe the physiological condition in mammals that is typically characterized by unregulated cell growth.

Examples of cancer include but are not limited to, carcinoma, lymphoma, sarcoma, blastema and leukemia. More particular examples of such cancers include squamous cell carcinoma, lung cancer, including non-small cell lung cancer, pancreatic cancer, cervical cancer, bladder cancer, hepatoma, breast cancer, colon carcinoma, including colorectal cancer, kidney cancer, including renal cell carcinoma and head and neck cancer, including Glioblastoma Multiforme (GBM), prostate cancer including CRPC, and bone cancer, including osteoblastic bone metastasis.

A VEGFR inhibitor is defined as a compound that inhibits the receptor as shown with in vitro testing or by other means. VEGF inhibitors include the following compound and compositions:

Aflibercept (also known as: AVE 0005, AVE 005, AVE0005; Bayer Healthcare/Sanofi-Aventis);

apatinib (also known as: YN-968D1, YN968D1; Advenchen, Inc.);

axitinib (also known as: AG-13736, AG-013736, Agouron/Pfizer);

bevacizumab (also known as: AVASTIN, R 435, R435, RG435; Genentech);

BIBF-1120 (also known as: Vargatef, Boehringer Ingelheim);

brivanib (also known as: BMS-582664, BMS-540215, IDDBCPl 80722; Bristol-Myers Squibb) Co);

semaxinib (also known as SU5416);

cediranib (also known as: RECENTIN, AZD-2171; AstraZeneca pic);

fluocinolone (also known as: MEDIDUR; ILUVIEN; Alimera Sciences Inc.);

linifanib (also known as: ABT-869, HT-1080, RG-3635, RG3635; Hoffmann-La Roche);

lapatinib+pazopanib (also known as: TYKERB+ARMALA, GlaxoSmithKline);

midostaurin (also known as: 4-N benzoylstaurosporine, 4-N-benzoyl staurosporine;

Benzoylstaurosporine, CGP 41251, N-benzoyl-staurosporine, PKC412, PKC412A; Novartis);

motesanib (also known as: AMG-706; Amgen, Inc.);

OTS-102 (OncoTherapy Science, Inc.);

AE-941 (also known as: Neovastat; Aeterna Laboratories);

pazopanib (also known as: GW-786034, VOTRIENT, ARMALA, 786034, GW-786034B; GlaxoSmithKline);

alacizumab pegol, BMS-690514;

pegaptanib (also known as: Macuverse (Macugen);

EYE-OOl (OcuPhor);

(OSI; Eyetech/IOMED) NX-1838);

ramucirumab (also known as: IMC-2C6, IMC-1121, IMC-1121B; ImClone Systems Inc.);

ranibizumab (also known as: Y0317, LUCENTIS, RG-3645; Genentech, Inc., Novartis, Inc);

ridoforolimus (also known as: AP-23573, AP-573, Ariad573, deforolimus, MK-8669; Ariad/Merck &amp; Co);

sorafenib (also known as: BAY-43-9006; IDDBCP150446, NEXAVAR, BAY-54-9085, Bayer AG, Onyx Pharmaceuticals, Inc.);

sunitinib (also known as: sutene, PHA-290940AD, SU-010398, SU-Ol 1248, SU-11248J, SU-12662, SUTENT, SU-11248; SUGEN Inc./Pfizer Inc., Pharmacia Corp.);

tivozanib (also known as: KRN-951, AV-951, AVEO Pharmaceuticals Inc);

vandetanib (also known as: AZD6474, ZACTIMA, ZD6474; AstraZeneca pic);

VEGF-Trap-Eye (Bayer);

SU4312 (Tocris Bioscience);

AEE-788 (Novartis) (also called AE-788 and NVP-AEE-788, among others);

AG-028262 (Pfizer);

AVE-8062 (Ajinomoto Co. and Sanofi-aventis);

BMS-3 87032 (Sunesis and Bristol-Myers Squibb);

CEP-7055 (Cephalon and Sanofi-aventis);

CHIR-258 (Chiron);

CP-547632 (OSI Pharmaceuticals and Pfizer);

CP-564959;

E-7080 (Eisai Co.);

GW-654652 (GlaxoSmithKline);

KRN-95 1 (Kirin Brewery Co.);

PKC-412 (Novartis);

PTK-787 (Novartis and Schering);

SU1 1248 (Sugen and Pfizer) (also called SU-1 1248, SU-Ol 1248, SU-1 1248J, SUTENT®, and sunitinib malate, among others);

SU-5416 (Sugen and Pfizer/Pharmacia) (also called CAS Registry Number 194413-58-6, semaxanib, 204005-46-9, among others);

SU-6668 (Sugen and Taiho) (also called CAS Registry Number 252916-29-3, SU-006668, and TSU-68, among others);

Thalidomide (Celgene) (also called CAS Registry Number 50-35-1, Synovir, Thalidomide Pharmion, and Thalomid, among others);

ZD-6474 (AstraZeneca) (also called CAS Registry Number 443913-73-3, Zactima, and AZD-6474, among others);

ZK-304709 (Schering) (also called CDK inhibitors (indirubin derivatives), ZK-CDK, MTGI, and multi-target tumor growth inhibitor, among others) and other closely related compounds including the indirubin derivative VEGF inhibitors described in WO 00/234717, WO 02/074742, WO 02/100401, WO 00/244148, WO 02/096888, WO 03/029223, WO 02/092079, and WO 02/094814.

VEGF inhibitors also include CDP791, Enzastaurin, Boehringer Ingelheim BIBF 1120, BAY 573952, BAY 734506, IMC-1 121B, CEP 701, SU 014813, SU 10944, SU 12662, OSI-930, and BMS 582664, and closely related VEGF inhibitors.

In addition to the foregoing inhibitors that act directly on VEGF or VEGFR, the following inhibitors have anti-angiogenic properties: ZD-6126 (AstraZeneca and Angiogene) (CAS Registry Number 219923-05-4, N-acetylcolchinol phosphate, ANG-453, AZD-6126, ZD-6126 derivatives and ZM-445526, among others) and closely related VEGF inhibitors such as other inhibitors in the ANG-400 series; Imatinib (Novartis) (CAS Registry Numbers 152459-95-5 and 220127-57-1, Glivec, Gleevec, STI-571, and CGP-57148, among others) and closely related VEGF inhibitors; RAD-001 (Novartis) (also called CAS Registry Number 159351-69-6, RAD-001, SDZ-RAD, Certican, and everolimus, among others) and closely related VEGF inhibitors; and BMS-354825 (Bristol-Myers Squibb) (CAS Registry Number 302962-49-8, Src/Abl kinase inhibitor, and dasatinib, among others) and closely related VEGF inhibitors.

Also useful in the invention in this are regard are CCl-779, 17-AAG, DMXAA, CI-1040, and CI-1033.

The following are also VEGF inhibitors: (a) a compound described in US 2003/0125339; (b) a substituted alkylamine derivative described in US 2003/0125339 or US 2003/0225106; (c) a substituted omega-carboxyaryl diphenyl urea or derivative thereof as described in WO 00/42012, WO 00/41698, US 2005/003 8080A1, US 2003/0125359A1, US 2002/0 165394A1, US 2001/003447A1, US 2001/0016659A1, and US 2002/013774A1; and (d) an anilinophthalazine or derivative thereof that binds to and inhibits the activity of multiple receptor tyrosine kinases including binding to the protein kinase domain and inhibition of VEGFR1 and VEGFR2.

Certain of the VEGF inhibitors are further described below, (1) motesanib; (2) NEXAVAR; (3) AZD-2171; (4) AG-13736; (5) AVASTIN; (6) PTK/ZK; and (7) SUTENT.

“Nexavar®” (also known as BAY 43-9006, sorafenib, CAS Registry Number 284461-73-0, raf kinase inhibitor, sorafenib analogs, and EDDBCP150446, among others) is a substituted omega carboxy diphenyl urea that inhibits RAF-I activation, and thereby decreases RAF-I dependent phosphorylation of MEK-I and ERK-I, as described in US Patent Application No. 2003/0125359A1, WO 03/047523A2, and Wilhelm et al, Current Pharmaceutical Design, 8:2255-2257 (2002), particularly relating to Nexavar®, its structure and properties, methods for making and using it, and other related molecules. A variety of derivatives have been produced. Among these are fluorinated derivatives described in US Patent Application 2005/0038080 A1 and WO 2005/009961, particularly as to these and other pharmaceutically active diphenyl urea compounds.

“PTK/ZK” also known as vatalanib, a multi-VEGF receptor Tyrosine kinase inhibitor that is said to block tumor angiogenesis and lymphangio genesis. Its chemical name is N-(4-chlorophenyl)-4-(pyridin-4-ylmethyl)phthalazin-1-amine. It also is known as CAS Registry Numbers 212141-54-3 and 212142-18-2, PTK787, PTK787/ZK, PTK-787/ZK-222584, PTK787/ZK222584, ZK-22584, VEGF-TKI, VEGF-RKI, PTK-787A, DE-00268, CGP-79787, CGP-79787D, vatalanib, and ZK-222584. See Thomas, A., et al., J. of Clin. Oncology, 23(18): 4162-4171 (2005); US Patent Application 2005/0118600A1, which are herein incorporated by reference in their entirety, particularly as to the structure, synthesis, properties, and uses of PTK/ZK and related compounds.

“Sutent®” is a small molecule receptor tyrosine kinase inhibitor with the chemical name (5-[5-fluoro-2-oxo-1,2-dihydroindol-(3Z)-ylidenemethyl]-2,4-dimethyl-1H-pyrrole-3-carboxylic acid [2-diethylaminoethyl]amide). Sutent® is also known as sunitinib malate, SU11248, SU-11248, SU-011248, and SU-11248J, and is reported to have anti-angiogenic and anti-tumor activities. See Mendel, D., et al., Clinical Cancer Research, 9:327-337 (2003); Schlessinger, J., The Scientist, 19(7): 17 (2005), which are herein incorporated by reference in their entirety, particularly as to the structure, synthesis, properties, and uses of Sutent® and related compounds.

“Avastin®,” also known as bevacizumab, is a recombinant humanized antibody to VEGF that binds to and inhibits VEGF.

“Motesanib” (AMG 706) is a multi-kinase inhibitor that interferes with the Kit, Ret, PDGF, and VEGF-signaling pathways, as described in U.S. Pat. No. 6,995,162, which is herein, incorporated by reference in its entirety, particularly in parts pertinent to motesanib, its structure and properties, methods for making and using it, and other related compounds. Its chemical name is N-(2,3-dihydro-3,3-dimethyl-1H-indol-6-yl)-2-[(4-pyridinylmethyl) amino]-3-pyridinecarboxamide. As used herein the term motesanib includes pharmaceutically acceptable salts, in particular, the diphosphate salt, except as otherwise provided herein.

An HGF/SF:MET inhibitor is defined as any small molecule (i.e., a compound with a molecular weight less than about 1000) or large molecule (i.e., a protein such as an antibody or antigen binding fragment) that interferes with the binding between HGF/SF and MET or otherwise blocks the kinase activity of MET, as shown with in vitro testing or by other means.

The following are among specific MET inhibitors that are contemplated in the invention: Amgen Compound 2 (1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide) is a selective MET inhibitor, as described in WO 2006/116713, which is herein incorporated by reference in its entirety, particularly in parts pertinent to Amgen Compound 2 as it relates to its structure and properties, methods for making and using them, and other related compounds, including pharmaceutically acceptable salts.

Amgen Compound 3 (N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide) is a selective MET inhibitor, as described in WO 2006/116713, particularly in parts pertinent to Amgen Compound 3, its structure and properties, methods for making and using.

PF-2341066 (Pfizer) including formulations for oral administration and closely related MET inhibitors;

PF042 17903 (Pfizer) including formulations for oral administration and closely related MET inhibitors;

ARQ197 (ArQule) including formulations for oral administration and closely related c-Met inhibitors;

MK2461 (Merck) including formulations for oral administration and closely related c-Met inhibitors;

MK8033 (Merck) including formulations for oral administration and closely related c-Met inhibitors;

ARQ 197 (ArQule) including formulations for oral administration and closely related c-Met inhibitors;

MGCD265 (Methylgene) including formulations for oral administration and closely related MET inhibitors;

JNJ38877605 (Johnson & Johnson) including formulations for oral administration and closely related MET inhibitors;

BMS777607 (Bristol Myers Squibb) including formulations for oral administration and closely related MET inhibitors;

E7050 (Eisai) including formulations for oral administration and closely related MET inhibitors;

MP-470 (SuperGen) including formulations for oral administration and closely related MET inhibitors; Compound X (N-[4-(6,7-dimethoxyquinolin-4-yloxy)-3-fluorophenyl]-N-phenylactylthiourea), as claimed in US 2004/0242603. Compound X includes pharmaceutically acceptable salts, as well as formulations for oral administration and closely related MET inhibitors; and

OA-5d5 (Genentech) (also called One Armed 5d5, 5d5, MetMab, PRO143966, among others) including formulations for oral administration and closely related MET inhibitors. OA-5d5 is a humanized anti-MET antibody, as described in US 2007/0092520.

An HGF/SF inhibitor is defined as a small molecule or large molecule that interferes with the binding between HGF/SF and MET by binding to and neutralizing HGF/SF, as shown with in vitro testing or by other means.

An anti-HGF/SF antibody is defined as an antibody, or fragment thereof, that interferes with the binding between HGF/SF and MET by binding to and neutralizing HGF/SF, as shown with in vitro testing or by other means, such as AMG 102 or L2G7 (Takeda-Galaxy Biotech).

1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (Amgen Compound 2),

N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide (Amgen Compound 3), ARQ197, MK2461, MK 8033, PF04217903, PF2341066, JNJ38877605, MGCD265, BMS 777607, AMG 458, INCB28060, AM7, and E7050.

Also included are combinations with monoclonal hepatocyte growth factor/scatter factor (HGF/SF):MET antibodies and fragments of HGF/SF:MET monoclonal antibodies, such as AV299, L2G7, OA-5d5 and AMG 102, or those described in U.S. Pat. No. 5,646,036 and U.S. Pat. No. 5,686,292.

Also included are combinations with humanized or fully human HGF/SF:c-Met antibodies, such as those described in US 2005/0118643, WO 2005/017107, US 2007/0092520, WO 2005/107800, WO 2007/115049, and U.S. Pat. No. 7,494,650 and U.S. Pat. No. 7,220,410.

To date, several possible MET inhibitors have been developed with the intent on either silencing, or decreasing MET expression or decreasing MET activity. For example, PHA665752 (Pfizer, Inc.), SUI 1274 (Sugen, Inc.), SUI 1271 (Sugen, Inc.), SUI 1606 (Sugen, Inc.), ARQ197 (ArQuleArqule, Inc.), MP470 (Supergen, Inc.), Kirin, Geldanamycins, SGX523 (SGX, Inc.), HPK-56 (Supergen, Inc.), AMGI 02 (Amgen, Inc.), MetMAb (Genentech, Inc.), ANG-797 (Angion Biomedica Corp.), CGEN-241 (Compugen LTD.), Metro-F-1 (Dompe S.p.A.), ABT-869 (Abbott Laboratories) and K252a are all MET inhibitors currently being produced.

### EMBODIMENTS

In one embodiment, the compound of Formula I is the compound of Formula Ia:

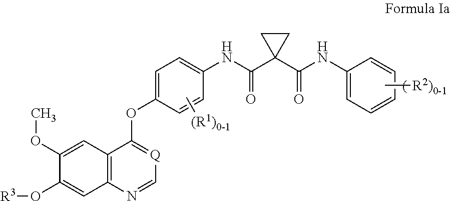

or a pharmaceutically acceptable salt thereof, wherein:

R¹ is halo;

R² is halo;

R³ is (C₁-C₆)alkyl or (C₁-C₆)alkyl optionally substituted with heterocycloalkyl; and

Q is CH or N.

In another embodiment, the compound of Formula I is the compound of Formula Ib:

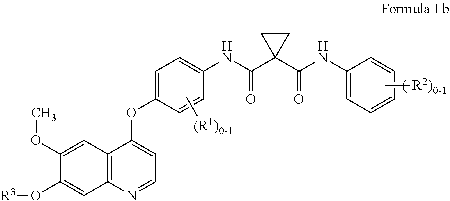

or a pharmaceutically acceptable salt thereof, wherein:

R¹ is halo;

R² is halo; and

R³ is (C₁-C₆)alkyl or (C₁-C₆)alkyl optionally substituted with heterocycloalkyl.

In another embodiment, the compound of Formula I is Compound 1.

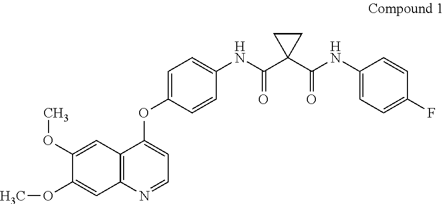 [SMILES: COc1cc2nccc(Oc3ccc(NC(=O)C4(C(=O)Nc5ccc(F)cc5)CC4)cc3)c2cc1OC]

In another embodiment, the compound of Formula I is Compound 2.

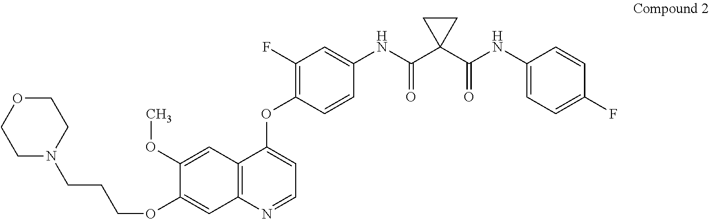 [SMILES: COc1cc2c(Oc3ccc(NC(=O)C4(C(=O)Nc5ccc(F)cc5)CC4)cc3F)ccnc2cc1OCCCN1CCOCC1]

In one embodiment, the compound of Formula II is the compound of Formula IIa:

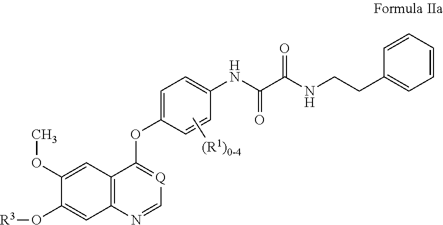

or a pharmaceutically acceptable salt thereof, wherein:

Q is CH or N;

R¹ is halo;

R² is phenyl; and

R³ is (C₁-C₆)alkyl substituted with heterocycloalkyl.

In another embodiment, the compound of Formula II is the compound of Formula IIb:

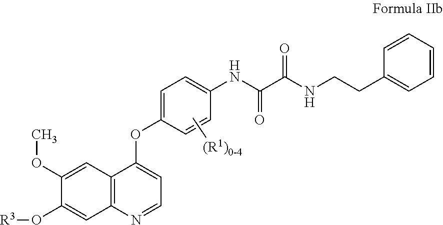

or a pharmaceutically acceptable salt thereof, wherein:

R¹ is halo;

R² is phenyl; and

R³ is (C₁-C₆)alkyl substituted with heterocycloalkyl.

In another embodiment, the compound of Formula II is Compound 3.

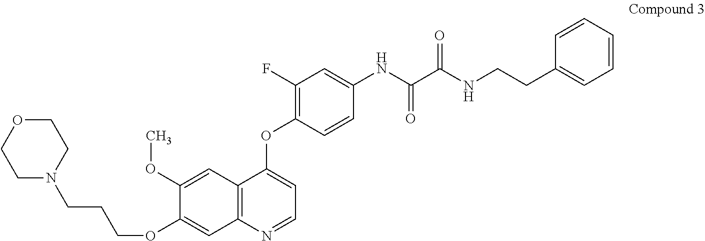 [SMILES: COc1cc2c(Oc3ccc(NC(=O)C(=O)NCCc4ccccc4)cc3F)ccnc2cc1OCCCN1CCOCC1]

In other embodiments, the compound of Formula I, Ia, Ib, II, IIa, IIb, Compound 1, Compound 2, or Compound 3, or a pharmaceutically acceptable salt thereof, is administered as a pharmaceutical composition, wherein the pharmaceutical composition comprises the compounds of Formula I, Ia, Ib, II, IIa, IIb, Compound 1, Compound 2, or Compound 3 and a pharmaceutically acceptable carrier, excipient, or diluent.

The compound of Formula I, Ia, Ib, II, IIa, IIb, Compound 1, Compound 2, or Compound 3, as described herein, includes both the recited compounds as well as individual isomers and mixtures of isomers. In each instance, the compound of Formula (I) includes the pharmaceutically acceptable salts, hydrates, and/or solvates of the recited compounds and any individual isomers or mixture of isomers thereof.

In other embodiments, the compound of Formula I is Compound 1 as the malate salt. The malate salt of Compound 1 is disclosed in PCT/US2010/021194 and 61/325,095.

In other embodiments, the compound of Formula I is Compound 2 as the crystalline hydrate form. The crystalline hydrate form is disclosed in 61/313,192, the entire contents of which is incorporated herein by reference.

In other embodiments, the compound of Formula II is Compound.

In another embodiment, the invention is directed to a method for ameliorating the symptoms of osteoblastic bone metastases, comprising administering to a patient in need of such treatment a therapeutically effective amount of a compound of Formula I or II in any of the embodiments disclosed herein.

In one aspect, the invention provides a method for treating bone cancer, prostate cancer, or bone cancer associated with prostate cancer, comprising administering a composition comprising:

(a) one or more inhibitor(s) of at least one of VEGF and VEGFR; and

(b) one or more inhibitor(s) of MET

to a patient in need of such treatment.

In one embodiment of this aspect, an inhibitor of at least one of VEGF and VEGFR is chosen from the group consisting of: aflibercept, apatinib, axitinib, bevacizumab, BIBF-1120, brivanib, semaxinib, cediranib, fluocinolone, lapatinib, lapatinib+pazopanib, linifanib, midostaurin, motesanib, OTS-102, AE-941, pazopanib, alacizumab pegol, BMS-690514, pegaptanib, EYE-001, ramucirumab, ranibizumab, ridoforolimus, sorafenib, sunitinib, tivozanib, vandetanib, VEGF-Trap-Eye, SU4312, Imatinib, Erlotinib, Gefitinib, Sorafenib, Sunitinib, Dasatinib, Vatalanib, LY294002, AEE-788, AG-028262, AVE-8062, BMS-3 87032, CEP-7055, CHIR-258, CP-547632, CP-564959, E-7080, GW-654652, KRN-95 1, PKC-412, PTK-787, SUE 1248, SU-5416, SU-6668, Thalidomide, ZD-6474, ZK-304709, CDP791, Enzastaurin, BIBF 1120, BAY 573952, BAY 734506, IMC-1 121B, CEP 701, SU 014813, SU 10944, SU 12662, OSI-930, and BMS 582664.

In a further embodiment, the inhibitor is a monoclonal antibody inhibitor chosen from Ranibizumab and Bevacizumab.

In one embodiment, the inhibitor of MET is chosen from the group consisting of 1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide, N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide, ARQ197, MK2461, MK 8033, PF04217903, PF2341066, JNJ38877605, MGCD265, BMS 777607, E7050, AV299, L2G7, OA-5d5, AMG 102, PHA665752, SU11274, SU11271, SU11606, ARQ197, MP470, Kirin, Geldanamycins, SGX523, HPK-56, MetMAb, ANG-797, CGEN-241, Metro-F-1, ABT-869, AMG 458, INCB28060, AM7, and K252a.

In a further embodiment, an inhibitor of MET is a monoclonal HGF/SF:MET antibody or a fragment of HGF/SF:MET monoclonal antibodies chosen from AV299, L2G7, OA-5d5 and AMG 102.

In still a further embodiment, an inhibitor of MET is the human monoclonal HGF/SF:MET antibody AMG 102.

In another embodiment, the prostate cancer is CRPC.

In another embodiment, the bone cancer is osteoblastic bone metastasis.

### Administration

Administration of the compound of Formula I, Ia, Ib, II, IIb, IIb, Compound 1, Compound 2, or Compound 3, or a pharmaceutically acceptable salt thereof, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin dosages (which can be in capsules or tablets), powders, solutions, suspensions, or aerosols, or the like, specifically in unit dosage forms suitable for simple administration of precise dosages.

The compositions will include a conventional pharmaceutical carrier or excipient and a compound of Formula I or II as the/an active agent, and, in addition, may include carriers and adjuvants, and so on.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the compound of Formula I may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (t) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the compound of Formula I, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administration are, for example, suppositories that can be prepared by mixing the compound of Formula I, Ia, Ib, II, IIa, IIb, Compound 1, Compound 2, or Compound 3, with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of the compound of Formula I, Ia, Ib, II, IIa, IIb, Compound 1, Compound 2, or Compound 3, include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this disclosure.

Compressed gases may be used to disperse the compound of Formula I, Ia, Ib, II, IIa, IIb, Compound 1, Compound 2, or Compound 3, in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of Formula I, Ia, Ib, II, IIa, IIb, Compound 1, Compound 2, or Compound 3, or a pharmaceutically acceptable salt thereof, and 99% to 1% by weight of a suitable pharmaceutical excipient. In one example, the composition will be between about 5% and about 75% by weight of a compound as disclosed herein, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of Formula I, or a pharmaceutically acceptable salt thereof, for treatment of a disease-state in accordance with the teachings of this disclosure.

The compounds of this disclosure, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compound of Formula I, I(a), I(b), Compound 1, or Compound 2, can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

In other embodiments, the compound of Formula I, Ia, Ib, II, IIa, IIb, Compound 1, Compound 2, or Compound 3, can be administered to the patient concurrently with other cancer treatments. Such treatments include other cancer chemotherapeutics, hormone replacement therapy, radiation therapy, or immunotherapy, among others. The choice of the other therapy depends on a number of factors including the metabolic stability and length of action of the compound, the age, body weight, general health, sex, diet, mode and time of administration, rate of excretion, drug combination, the severity of the particular disease-states, and the host undergoing therapy.

### Preparation of the Compound 1

### Preparation of N-(4-{[6,7-bis(methyloxy)quinolin-4-yl]oxy}phenyl)-N′-(4-fluorophenyl)cyclopropane-1,1-dicarboxamide and the (L)-malate salt thereof

The synthetic route used for the preparation of N-(4-[6,7-bis(methyloxy)quinolin-4-yl]oxy}phenyl)-M-(4-fluorophenyl)cyclopropane-1,1-dicarboxamide and the (L)-malate salt thereof is depicted in Scheme 1:

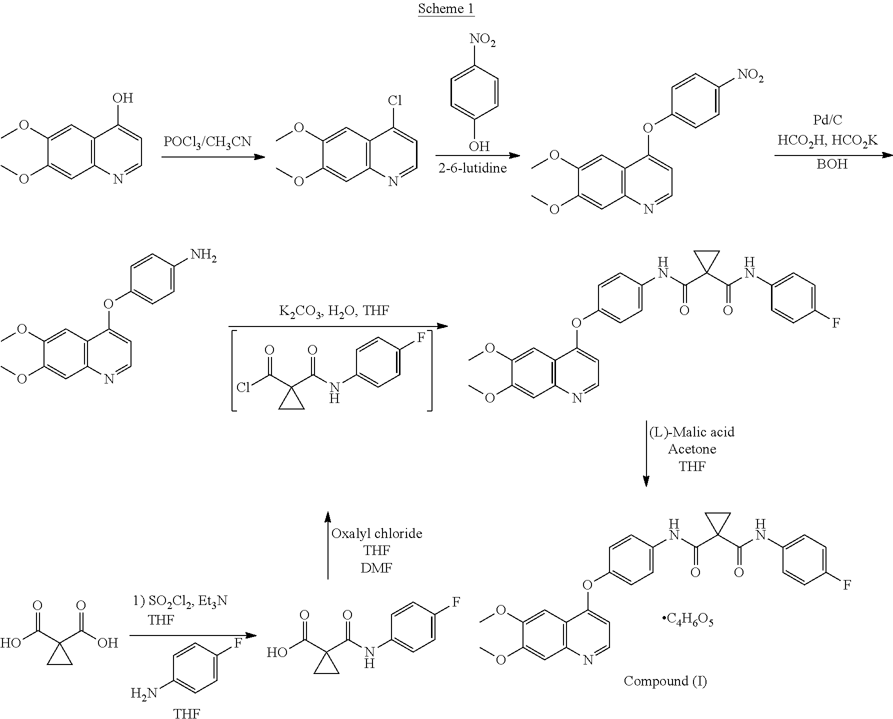

### Preparation of 4-Chloro-6,7-dimethoxy-quinoline

A reactor was charged sequentially with 6,7-dimethoxy-quinoline-4-ol (10.0 kg) and acetonitrile (64.0 L). The resulting mixture was heated to approximately 65° C. and phosphorus oxychloride (POCl₃, 50.0 kg) was added. After the addition of POCl₃, the temperature of the reaction mixture was raised to approximately 80° C. The reaction was deemed complete (approximately 9.0 hours) when less than 2 percent of the starting material remained (in process high-performance liquid chromotography [HPLC] analysis). The reaction mixture was cooled to approximately 10° C. and then quenched into a chilled solution of dichloromethane (DCM, 238.0 kg), 30% NH₄OH (135.0 kg), and ice (440.0 kg). The resulting mixture was warmed to approximately 14° C., and phases were separated. The organic phase was washed with water (40.0 kg) and concentrated by vacuum distillation to remove the solvent (approximately 190.0 kg). Methyl-t-butyl ether (MTBE, 50.0 kg) was added to the batch, and the mixture was cooled to approximately 10° C., during which time the product crystallized out. The solids were recovered by centrifugation, washed with n heptane (20.0 kg), and dried at approximately 40° C. to afford the title compound (8.0 kg).

### Preparation of 6,7-Dimethyl-4-(4-nitro-phenoxy)-quinoline

A reactor was sequentially charged with 4-chloro-6,7-dimethoxy-quinoline (8.0 kg), 4 nitrophenol (7.0 kg), 4 dimethylaminopyridine (0.9 kg), and 2,6 lutidine (40.0 kg). The reactor contents were heated to approximately 147° C. When the reaction was complete (less than 5 percent starting material remaining as determined by in process HPLC analysis, approximately 20 hours), the reactor contents were allowed to cool to approximately 25° C. Methanol (26.0 kg) was added, followed by potassium carbonate (3.0 kg) dissolved in water (50.0 kg). The reactor contents were stirred for approximately 2 hours. The resulting solid precipitate was filtered, washed with water (67.0 kg), and dried at 25° C. for approximately 12 hours to afford the title compound (4.0 kg).

### Preparation of 4-(6,7-Dimethoxy-quinoline-4-yloxy)-phenylamine

A solution containing potassium formate (5.0 kg), formic acid (3.0 kg), and water (16.0 kg) was added to a mixture of 6,7-dimethoxy-4-(4-nitro-phenoxy)-quinoline (4.0 kg), 10 percent palladium on carbon (50 percent water wet, 0.4 kg) in tetrahydrofuran (THF, 40.0 kg) that had been heated to approximately 60° C. The addition was carried out such that the temperature of the reaction mixture remained approximately 60° C. When the reaction was deemed complete as determined using in-process HPLC analysis (less than 2 percent starting material remaining, typically 15 hours), the reactor contents were filtered. The filtrate was concentrated by vacuum distillation at approximately 35° C. to half of its original volume, which resulted in the precipitation of the product. The product was recovered by filtration, washed with water (12.0 kg), and dried under vacuum at approximately 50° C. to afford the title compound (3.0 kg; 97 percent area under curve (AUC)).

### Preparation of 1-(4-Fluoro-phenylcarbamoyl)-cyclopropanecarboxylic acid

Triethylamine (8.0 kg) was added to a cooled (approximately 4° C.) solution of commercially available cyclopropane-1,1-dicarboxylic acid (21, 10.0 kg) in THF (63.0 kg) at a rate such that the batch temperature did not exceed 10° C. The solution was stirred for approximately 30 minutes, and then thionyl chloride (9.0 kg) was added, keeping the batch temperature below 10° C. When the addition was complete, a solution of 4-fluoroaniline (9.0 kg) in THF (25.0 kg) was added at a rate such that the batch temperature did not exceed 10° C. The mixture was stirred for approximately 4 hours and then diluted with isopropyl acetate (87.0 kg). This solution was washed sequentially with aqueous sodium hydroxide (2.0 kg dissolved in 50.0 L of water), water (40.0 L), and aqueous sodium chloride (10.0 kg dissolved in 40.0 L of water). The organic solution was concentrated by vacuum distillation followed by the addition of heptane, which resulted in the precipitation of solid. The solid was recovered by centrifugation and then dried at approximately 35° C. under vacuum to afford the title compound. (10.0 kg).

### Preparation of 1-(4-Fluoro-phenylcarbamoyl)-cyclopropanecarbonyl chloride

Oxalyl chloride (1.0 kg) was added to a solution of 1-(4-fluoro-phenylcarbamoyl)-cyclopropanecarboxylic acid (2.0 kg) in a mixture of THF (11 kg) and N,N-dimethylformamide (DMF; 0.02 kg) at a rate such that the batch temperature did not exceed 30° C. This solution was used in the next step without further processing.

### Preparation of N-(4-{[6,7-bis(methyloxy)quinolin-4-yl]oxy}phenyl)-N′-(4-fluorophenyl)cyclopropane-1,1-dicarboxamide

The solution from the previous step containing 1-(4-fluoro-phenylcarbamoyl)-cyclopropanecarbonyl chloride was added to a mixture of 4-(6,7-dimethoxy-quinoline-4-yloxy)-phenylamine (3.0 kg) and potassium carbonate (4.0 kg) in THF (27.0 kg) and water (13.0 kg) at a rate such that the batch temperature did not exceed 30° C. When the reaction was complete (in typically 10 minutes), water (74.0 kg) was added. The mixture was stirred at 15-30° C. for approximately 10 hours, which resulted in the precipitation of the product. The product was recovered by filtration, washed with a pre-made solution of THF (11.0 kg) and water (24.0 kg), and dried at approximately 65° C. under vacuum for approximately 12 hours to afford the title compound (free base, 5.0 kg). ¹H NMR (400 MHz, d₆-DMSO): δ 10.2 (s, 1H), 10.05 (s, 1H), 8.4 (s, 1H), 7.8 (m, 2H), 7.65 (m, 2H), 7.5 (s, 1H), 7.35 (s, 1H), 7.25 (m, 2H), 7.15 (m, 2H), 6.4 (s, 1H), 4.0 (d, 6H), 1.5 (s, 4H). LC/MS: 502.

### Preparation of N-(4-{([6,7-bis(methyloxy)quinolin-4-yl]oxy}phenyl)-N′-(4-fluorophenyl)cyclopropane-1,1-dicarboxamide, (L) malate salt

A solution of L-malic acid (2.0 kg) in water (2.0 kg) was added to a solution of Cyclopropane-1,1-dicarboxylic acid [4-(6,7-dimethoxy-quinoline-4-yloxy)-phenyl]-amide (4-fluoro-phenyl)-amide free base (15, 5.0 kg) in ethanol, maintaining a batch temperature of approximately 25° C. Carbon (0.5 kg) and thiol silica (0.1 kg) were then added, and the resulting mixture was heated to approximately 78° C., at which point water (6.0 kg) was added. The reaction mixture was then filtered, followed by the addition of isopropanol (38.0 kg), and was allowed to cool to approximately 25° C. The product was recovered by filtration and washed with isopropanol (20.0 kg), and dried at approximately 65° C. to afford the title compound (5.0 kg).

### Alternative Preparation of N-(4-{[6,7-Bis(methyloxy)quinolin-4-yl]oxy}phenyl)-N′-(4-fluorophenyl)cyclopropane-1,1-dicarboxamide and the (L)-malate salt thereof

An alternative synthetic route that can be used for the preparation of N-(4-{[6,7-bis(methyloxy)quinolin-4-yl]oxy}phenyl)-N′-(4-fluorophenyl)cyclopropane-1,1-dicarboxamide and the (L)-malate salt thereof is depicted in Scheme 2.

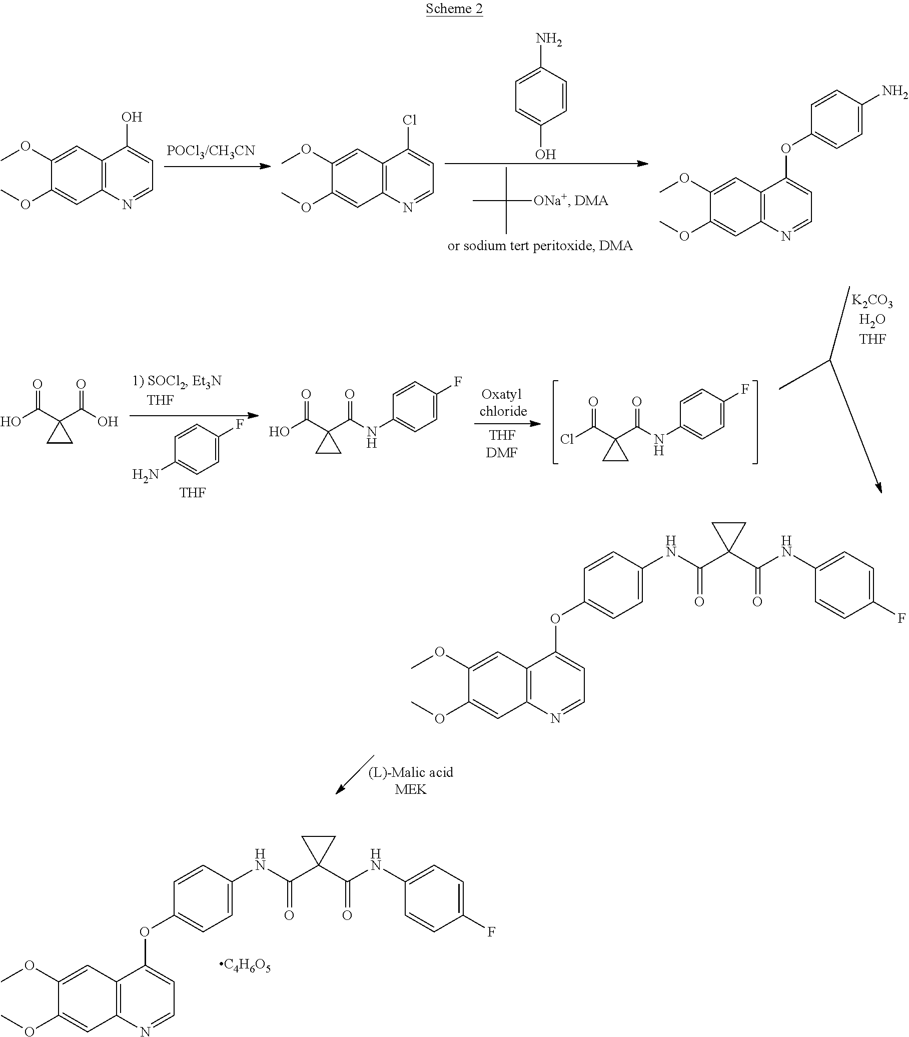

### Preparation of 4-Chloro-6,7-dimethoxy-quinoline

A reactor was charged sequentially with 6,7-dimethoxy-quinoline-4-ol (47.0 kg) and acetonitrile (318.8 kg). The resulting mixture was heated to approximately 60° C. and phosphorus oxychloride (POCl₃, 130.6 kg) was added. After the addition of POCl₃, the temperature of the reaction mixture was raised to approximately 77° C. The reaction was deemed complete (approximately 13 hours) when less than 3% of the starting material remained (in-process high-performance liquid chromatography [HPLC] analysis). The reaction mixture was cooled to approximately 2-7° C. and then quenched into a chilled solution of dichloromethane (DCM, 482.8 kg), 26 percent NH₄OH (251.3 kg), and water (900 L). The resulting mixture was warmed to approximately 20-25° C., and phases were separated. The organic phase was filtered through a bed of AW hyflo super-cel NF (Celite; 5.4 kg) and the filter bed was washed with DCM (118.9 kg). The combined organic phase was washed with brine (282.9 kg) and mixed with water (120 L). The phases were separated and the organic phase was concentrated by vacuum distillation with the removal of solvent (approximately 95 L residual volume). DCM (686.5 kg) was charged to the reactor containing organic phase and concentrated by vacuum distillation with the removal of solvent (approximately 90 L residual volume). Methyl t-butyl ether (MTBE, 226.0 kg) was then charged and the temperature of the mixture was adjusted to −20 to −25° C. and held for 2.5 hours resulting in solid precipitate which was then filtered and washed with n-heptane (92.0 kg), and dried on a filter at approximately 25° C. under nitrogen to afford the title compound. (35.6 kg).

### Preparation of 4-(6,7-Dimethoxy-quinoline-4-yloxy)-phenylamine

4-Aminophenol (24.4 kg) dissolved in N,N-dimethylacetamide (DMA, 184.3 kg) was charged to a reactor containing 4-chloro-6,7-dimethoxyquinoline (35.3 kg), sodium t-butoxide (21.4 kg) and DMA (167.2 kg) at 20-25° C. This mixture was then heated to 100-105° C. for approximately 13 hours. After the reaction was deemed complete as determined using in-process HPLC analysis (less than 2 percent starting material remaining), the reactor contents were cooled at 15-20° C. and water (pre-cooled, 2-7° C., 587 L) charged at a rate to maintain 15-30° C. temperature. The resulting solid precipitate was filtered, washed with a mixture of water (47 L) and DMA (89.1 kg) and finally with water (214 L). The filter cake was then dried at approximately 25° C. on filter to yield crude 4-(6,7-dimethoxy-quinoline-4-yloxy)-phenylamine (59.4 kg wet, 41.6 kg dry calculated based on LOD). Crude 4-(6,7-dimethoxy-quinoline-4-yloxy)-phenylamine was refluxed (approximately 75° C.) in a mixture of tetrahydrofuran (THF, 211.4 kg) and DMA (108.8 kg) for approximately 1 hour and then cooled to 0-5° C. and aged for approximately 1 hour after which time the solid was filtered, washed with THF (147.6 kg) and dried on a filter under vacuum at approximately 25° C. to yield 4-(6,7-dimethoxy-quinoline-4-yloxy)-phenylamine (34.0 kg).

### Alternative Preparation of 4-(6,7-Dimethoxy-quinoline-4-yloxy)-phenylamine

4-chloro-6,7-dimethoxyquinoline (34.8 kg) and 4-aminophenol (30.8 kg) and sodium tert pentoxide (1.8 equivalents) 88.7 kg, 35 weight percent in THF) were charged to a reactor, followed by N,N-dimethylacetamide (DMA, 293.3 kg). This mixture was then heated to 105-115° C. for approximately 9 hours. After the reaction was deemed complete as determined using in-process HPLC analysis (less than 2 percent starting material remaining), the reactor contents were cooled at 15-25° C. and water (315 kg) was added over a two hour period while maintaining the temperature between 20-30° C. The reaction mixture was then agitated for an additional hour at 20-25° C. The crude product was collected by filtration and washed with a mixture of 88 kg water and 82.1 kg DMA, followed by 175 kg water. The product was dried on a filter drier for 53 hours. The LOD showed less than 1 percent w/w.

In an alternative procedure, 1.6 equivalents of sodium tert-pentoxide were used and the reaction temperature was increased from 110-120° C. In addition, the cool down temperature was increased to 35-40° C. and the starting temperature of the water addition was adjusted to 35-40° C., with an allowed exotherm to 45° C.

### Preparation of 1-(4-Fluoro-phenylcarbamoyl)-cyclopropanecarboxylic acid

Triethylamine (19.5 kg) was added to a cooled (approximately 5° C.) solution of cyclopropane-1,1-dicarboxylic acid (24.7 kg) in THF (89.6 kg) at a rate such that the batch temperature did not exceed 5° C. The solution was stirred for approximately 1.3 hours, and then thionyl chloride (23.1 kg) was added, keeping the batch temperature below 10° C. When the addition was complete, the solution was stirred for approximately 4 hours keeping temperature below 10° C. A solution of 4-fluoroaniline (18.0 kg) in THF (33.1 kg) was then added at a rate such that the batch temperature did not exceed 10° C. The mixture was stirred for approximately 10 hours after which the reaction was deemed complete. The reaction mixture was then diluted with isopropyl acetate (218.1 kg). This solution was washed sequentially with aqueous sodium hydroxide (10.4 kg, 50 percent dissolved in 119 L of water) further diluted with water (415 L), then with water (100 L) and finally with aqueous sodium chloride (20.0 kg dissolved in 100 L of water). The organic solution was concentrated by vacuum distillation (100 L residual volume) below 40° C. followed by the addition of n-heptane (171.4 kg), which resulted in the precipitation of solid. The solid was recovered by filtration and washed with n-heptane (102.4 kg), resulting in wet, crude 1-(4-fluoro-phenylcarbamoyl)-cyclopropanecarboxylic acid (29.0 kg). The crude, 1-(4-fluoro-phenylcarbamoyl)-cyclopropanecarboxylic acid was dissolved in methanol (139.7 kg) at approximately 25° C. followed by the addition of water (320 L) resulting in slurry which was recovered by filtration, washed sequentially with water (20 L) and n-heptane (103.1 kg) and then dried on the filter at approximately 25° C. under nitrogen to afford the title compound (25.4 kg).

### Preparation of 1-(4-Fluoro-phenylcarbamoyl)-cyclopropanecarbonyl chloride

Oxalyl chloride (12.6 kg) was added to a solution of 1-(4-fluoro-phenylcarbamoyl)-cyclopropanecarboxylic acid (22.8 kg) in a mixture of THF (96.1 kg) and N,N-dimethylformamide (DMF; 0.23 kg) at a rate such that the batch temperature did not exceed 25° C. This solution was used in the next step without further processing.

### Alternative Preparation of 1-(4-Fluoro-phenylcarbamoyl)-cyclopropanecarbonyl chloride

A reactor was charged with 1-(4-fluoro-phenylcarbamoyl)-cyclopropanecarboxylic acid (35 kg), 344 g DMF, and 175 kg THF. The reaction mixture was adjusted to 12-17° C. and then to the reaction mixture was charged 19.9 kg of oxalyl chloride over a period of 1 hour. The reaction mixture was left stirring at 12-17° C. for 3 to 8 hours. This solution was used in the next step without further processing.

### Preparation of cyclopropane-1,1-dicarboxylic acid [4-(6,7-dimethoxy-quinoline-4-yloxy)-phenyl]-amide (4-fluoro-phenyl)-amide

The solution from the previous step containing 1-(4-fluoro-phenylcarbamoyl)-cyclopropanecarbonyl chloride was added to a mixture of compound 4-(6,7-dimethoxy-quinoline-4-yloxy)-phenylamine (23.5 kg) and potassium carbonate (31.9 kg) in THE (245.7 kg) and water (116 L) at a rate such that the batch temperature did not exceed 30° C. When the reaction was complete (in approximately 20 minutes), water (653 L) was added. The mixture was stirred at 20-25° C. for approximately 10 hours, which resulted in the precipitation of the product. The product was recovered by filtration, washed with a pre-made solution of THF (68.6 kg) and water (256 L), and dried first on a filter under nitrogen at approximately 25° C. and then at approximately 45° C. under vacuum to afford the title compound (41.0 kg, 38.1 kg, calculated based on LOD).

### Alternative Preparation of cyclopropane-1,1-dicarboxylic acid [4-(6,7-dimethoxy-quinoline-4-yloxy)-phenyl]-amide (4-fluoro-phenyl)-amide

A reactor was charged with 4-(6,7-dimethoxy-quinoline-4-yloxy)-phenylamine (35.7 kg, 1 equivalent), followed by 412.9 kg THF. To the reaction mixture was charged a solution of 48.3K₂CO₃ in 169 kg water. The acid chloride solution of described in the Alternative Preparation of 1-(4-Fluoro-phenylcarbamoyl)-cyclopropanecarbonyl chloride above was transferred to the reactor containing 4-(6,7-dimethoxy-quinoline-4-yloxy)-phenylamine while maintaining the temperature between 20-30° C. over a minimum of two hours. The reaction mixture was stirred at 20-25° C. for a minimum of three hours. The reaction temperature was then adjusted to 30-25° C. and the mixture was agitated. The agitation was stopped and the phases of the mixture were allowed to separate. The lower aqueous phase was removed and discarded. To the remaining upper organic phase was added 804 kg water. The reaction was left stirring at 15-25° C. for a minimum of 16 hours.

The product precipitated. The product was filtered and washed with a mixture of 179 kg water and 157.9 kg THF in two portions. The crude product was dried under a vacuum for at least two hours. The dried product was then taken up in 285.1 kg THF. The resulting suspension was transferred to reaction vessel and agitated until the suspension became a clear (dissolved) solution, which required heating to 30-35° C. for approximately 30 minutes. 456 kg water was then added to the solution, as well as 20 kg SDAG-1 ethanol

(ethanol denatured with methanol over two hours. The mixture was agitated at 15-25° C. fir at least 16 hours. The product was filtered and washed with a mixture of 143 kg water and 126.7 THF in two portions. The product was dried at a maximum temperature set point of 40° C.

In an alternative procedure, the reaction temperature during acid chloride formation was adjusted to 10-15° C. The recrystallization temperature was changed from 15-25° C. to 45-50° C. for 1 hour and then cooled to 15-25° C. over 2 hours.

### Preparation of cyclopropane-1,1-dicarboxylic acid [4-(6,7-dimethoxy-quinoline-4-yloxy)-phenyl]-amide (4-fluoro-phenyl)-amide, malate salt

Cyclopropane-1,1-dicarboxylic acid [4-(6,7-dimethoxy-quinoline-4-yloxy)-phenyl]-amide (4-fluoro-phenyl)-amide (1-5; 13.3 kg), L-malic acid (4.96 kg), methyl ethyl ketone (MEK; 188.6 kg) and water (37.3 kg) were charged to a reactor and the mixture was heated to reflux (approximately 74° C.) for approximately 2 hours. The reactor temperature was reduced to 50 to 55° C. and the reactor contents were filtered. These sequential steps described above were repeated two more times starting with similar amounts of starting material (13.3 kg), L-Malic acid (4.96 kg), MEK (198.6 kg) and water (37.2 kg). The combined filtrate was azeotropically dried at atmospheric pressure using MEK (1133.2 kg) (approximate residual volume 711 L; KF≦0.5% w/w) at approximately 74° C. The temperature of the reactor contents was reduced to 20 to 25° C. and held for approximately 4 hours resulting in solid precipitate which was filtered, washed with MEK (448 kg) and dried under vacuum at 50° C. to afford the title compound (45.5 kg).

### Alternative Preparation of cyclopropane-1,1-dicarboxylic acid [4-(6,7-dimethoxy-quinoline-4-yloxy)-phenyl]-amide (4-fluoro-phenyl)-amide, (L) malate salt

Cyclopropane-1,1-dicarboxylic acid [4-(6,7-dimethoxy-quinoline-4-yloxy)-phenyl]-amide (4-fluoro-phenyl)-amide (47.9 kg), L-malic acid (17.2), 658.2 kg methyl ethyl ketone, and 129.1 kg water (37.3 kg) were charged to a reactor and the mixture was heated 50-55° C. for approximately 1-3 hours, and then at 55-60° C. for an additional 4-5 hours. The mixture was clarified by filtration through a 1 μm cartridge. The reactor temperature was adjusted to 20-25° C. and vacuum distilled with a vacuum at 150-200 mm Hg with a maximum jacket temperature of 55° C. to the volume range of 558-731 L.

The vacuum distillation was performed two more times with the charge of 380 kg and 380.2 kg methyl ethyl ketone, respectively. After the third distillation, the volume of the batch was adjusted to 18 v/w of cyclopropane-1,1-dicarboxylic acid [4-(6,7-dimethoxy-quinoline-4-yloxy)-phenyl]-amide (4-fluoro-phenyl)-amide by charging 159.9 kg methyl ethyl ketone to give a total volume of 880 L. An additional vacuum distillation was carried out by adjusting 245.7 methyl ethyl ketone. The reaction mixture was left with moderate agitation at 20-25° C. for at least 24 hours. The product was filtered and washed with 415.1 kg methyl ethyl ketone in three portions. The product was dried under a vacuum with the jacket temperature set point at 45° C.

In an alternative procedure, the order of addition was changed so that a solution of 17.7 kg L-malic acid dissolved in 129.9 kg water was added to cyclopropane-1,1-dicarboxylic acid [4-(6,7-dimethoxy-quinoline-4-yloxy)-phenyl]-amide (4-fluoro-phenyl)-amide (48.7 kg) in methyl ethyl ketone (673.3 kg).

### Preparation of Compound 2

Compound 2 was prepared as provided in Scheme 3 and the accompanying experimental examples.

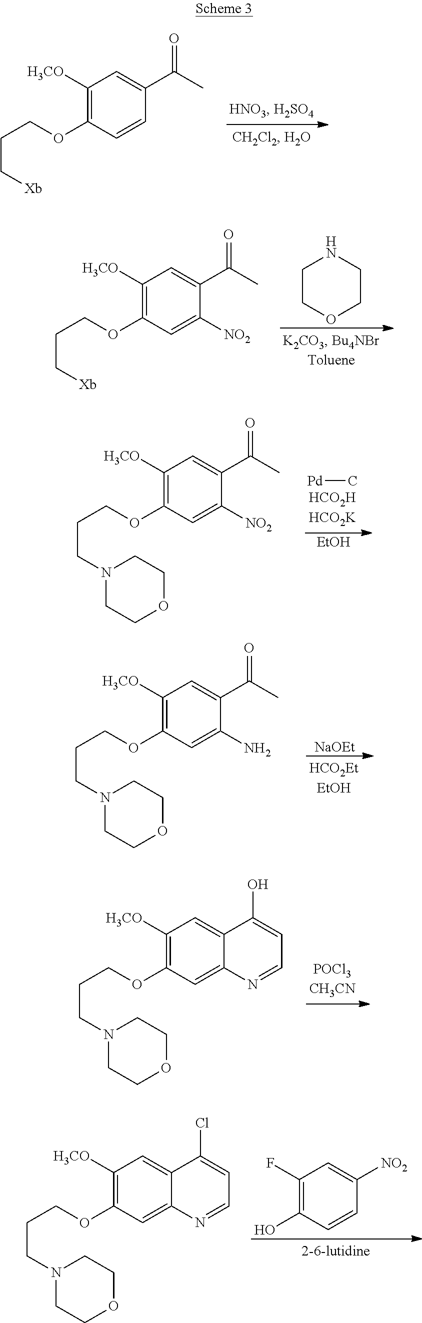

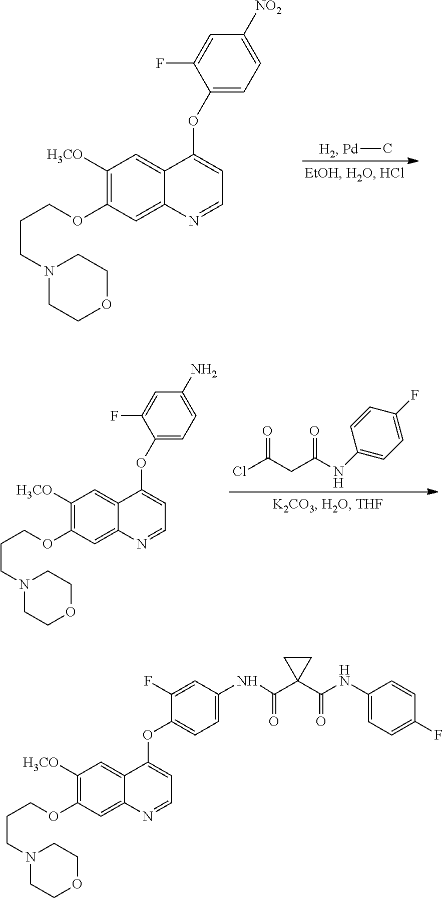

In Scheme 1, Xb is Br or Cl. For the names of the intermediates described within the description of Scheme 1 below, Xb is referred to as halo, wherein this halo group for these intermediates is meant to mean either Br or Cl.

### Preparation of 1-[5 methoxy-4 (3-halo propoxy)-2 nitro-phenyl]-ethanone

Water (70 L) was charged to the solution of 1-[4-(3-halo propoxy)-3-methoxy phenyl]ethanone (both the bromo and the chloro compound are commercially available). The solution was cooled to approximately 4° C. Concentrated sulfuric acid (129.5 kg) was added at a rate such that the batch temperature did not exceed approximately 18° C. The resulting solution was cooled to approximately 5° C. and 70 percent nitric acid (75.8 kg) was added at a rate such that the batch temperature did not exceed approximately 10° C. Methylene chloride, water and ice were charged to a separate reactor. The acidic reaction mixture was then added into this mixture. The methylene chloride layer was separated and the aqueous layer was back extracted with methylene chloride. The combined methylene chloride layers were washed with aqueous potassium bicarbonate solution and concentrated by vacuum distillation. 1-Butanol was added and the mixture was again concentrated by vacuum distillation. The resulting solution was stirred at approximately 20° C. during which time the product crystallized. The solids were collected by filtration, washed with 1-butanol to afford compound the title compound, which was isolated as a solvent wet cake and used directly in the next step. ¹HNMR (400 MHz, DMSO-d6): δ 7.69 (s, 1H), 7.24 (s, 1H); 4.23 (m, 2H), 3.94 (s, 3H), 3.78 (t)-3.65 (t) (2H), 2.51 (s, 3H), 2.30-2.08 (m, 2H) LC/MS Calcd for [M(Cl)+H]⁺288.1. found 288.0; Calcd for [M(Br)+H]⁺332.0, 334.0. found 331.9, 334.0.

### Preparation of 1-[5-methoxy-4-(3-morpholin-4-yl-propoxy)-2-nitro-phenyl]-ethanone

The solvent wet cake isolated in the previous step was dissolved in toluene. A solution of sodium iodide (67.9 kg) and potassium carbonate (83.4 kg) was added to this solution, followed by tetrabutylammonium bromide (9.92 kg) and morpholine (83.4 kg). The resulting 2 phase mixture was heated to approximately 85° C. for about 9 hours. The mixture was then cooled to ambient temperature. The organic layer was removed. The aqueous layer was back extracted with toluene. The combined toluene layers were washed sequentially with two portions of saturated aqueous sodium thiosulfate followed by two portions of water. The resulting solution of the title compound was used in the next step without further processing. ¹HNMR (400 MHz, DMSO-d6): δ 7.64 (s, 1H), 7.22 (s, 1H), 4.15 (t, 2H), 3.93 (s, 3H), 3.57 (t, 4H), 2.52 (s, 3H), 2.44-2.30 (m, 6H), 1.90 (quin, 2H); LC/MS Calcd for [M+H]⁺339.2. found 339.2.

### Preparation of 1-[2-amino-5-methoxy-4-(3-morpholin-4-yl-propoxy)-phenyl]-ethanone

The solution from the previous step was concentrated under reduced pressure to approximately half of the original volume. Ethanol and 10 percent Pd C (50 percent water wet, 5.02 kg) were added; the resulting slurry was heated to approximately 48° C. and an aqueous solution of formic acid (22.0 kg) and potassium formate (37.0 kg) was added. When the addition was complete and the reaction deemed complete by thin layer chromatography (TLC), water was added to dissolve the by-product salts. The mixture was filtered to remove the insoluble catalyst. The filtrate was concentrated under reduced pressure and toluene was added. The mixture was made basic (pH of about 10) by the addition of aqueous potassium carbonate. The toluene layer was separated and the aqueous layer was back extracted with toluene. The combined toluene phases were dried over anhydrous sodium sulfate. The drying agent was removed by filtration and the resulting solution was used in the next step without further processing. ¹HNMR (400 MHz, DMSO-d6): δ 7.11 (s, 1H), 7.01 (br s, 2H), 6.31 (s, 1H), 3.97 (t, 2H), 3.69 (s, 3H), 3.57 (t, 4H), 2.42 (s, 3H), 2.44-2.30 (m, 6H), 1.91 (quin, 2H LC/MS Calcd for [M+H]⁺309.2. found 309.1.

### Preparation of 6-methoxy-7-(3-morpholin-4-yl-propoxy)-quinolin-4-ol, sodium salt

A solution of sodium ethoxide (85.0 kg) in ethanol and ethyl formate (70.0 kg) was added to the solution from the previous step. The mixture was warmed to approximately 44° C. for about 3 hours. The reaction mixture was cooled to approximately 25° C. Methyl t-butyl ether (MTBE) was added which caused the product to precipitate. The product was collected by filtration and the cake was washed with MTBE and dried under reduced pressure at ambient temperature. The dried product was milled through a mesh screen to afford 60.2 kg of the title compound. ¹HNMR (400 MHz, DMSO-d6): δ 11.22 (br s, 1H), 8.61 (d, 1H), 7.55 (s, 1H), 7.54 (s, 1H), 7.17 (d, 1H), 4.29 (t, 2H), 3.99 (m, 2H), 3.96 (s, 3H), 3.84 (t, 2H), 3.50 (d, 2H), 3.30 (m, 2H), 3.11 (m, 2H), 2.35 (m, 2H), LC/MS Calcd for [M+H]⁺319.2. found 319.1.

### Preparation of 4-chlor-6-methoxy-7-(3 morpholin-4-yl)-quinoline

Phosphorous oxychloride (26.32 kg) was added to a solution of 6-methoxy-7-(3-morpholin-4-yl-propoxy)-quinolin-4-ol (5.00 kg) in acetonitrile that was heated to 50-55° C. When the addition was complete, the mixture was heated to reflux (approximately 82° C.) and held at that temperature, with stirring for approximately 18 hours at which time it was sampled for in process HPLC analysis. The reaction was considered complete when no more than 5 percent starting material remained. The reaction mixture was then cooled to 20-25° C. and filtered to remove solids. The filtrate was then concentrated to a residue. Acetronitrile was added and the resulting solution was concentrated to a residue. Methylene chloride was added to the residue and the resulting solution was quenched with a mixture of methylene chloride and aqueous ammonium hydroxide. The resulting 2 phase mixture was separated and the aqueous layer was back extracted with methylene chloride. The combined methylene chloride solutions were dried over anhydrous magnesium sulfate, filtered and concentrated to a solid. The solids were dried at 30-40° C. under reduced pressure to afford the title compound (1.480 kg). ¹HNMR (400 MHz, DMSO-d6): δ 8.61 (d, 1H), 7.56 (d, 1H), 7.45 (s, 1H), 7.38 (s, 1H), 4.21 (t, 2H), 3.97 (s, 3H), 3.58 (m, 2H), 2.50-2.30 (m, 6H), 1.97 (quin, 2H) LC/MS Calcd for [M+H]⁺458.2. found 458.0.

### Preparation of 4-(2-fluoro-4-nitro-phenoxy)-6-methoxy-7-(3-morpholin-4-yl propoxy)quinoline

A solution of 4-chloro-6-methoxy-7-(3 morpholin-4-yl)-quinoline (2.005 kg, 5.95 mol) and 2 fluoro-4-nitrophenol (1.169 kg, 7.44 mol) in 2,6-lutidine was heated to 140-145° C., with stirring, for approximately 2 hours, at which time it was sampled for in process HPLC analysis. The reaction was considered complete when less than 5 percent starting material remained. The reaction mixture was then cooled to approximately 75° C. and water was added. Potassium carbonate was added to the mixture, which was then stirred at ambient temperature overnight. The solids that precipitated were collected by filtration, washed with aqueous potassium carbonate, and dried at 55-60° C. under reduced pressure to afford the title compound (1.7 kg). ¹HNMR (400 MHz, DMSO-d6): δ 8.54 (d, 1H), 8.44 (dd, 1H), 8.18 (m, 1H), 7.60 (m, 1H), 7.43 (s, 1H), 7.42 (s, 1H), 6.75 (d, 1H), 4.19 (t, 2H), 3.90 (s, 3H), 3.56 (t, 4H), 2.44 (t, 2H), 2.36 (m, 4H), 1.96 (m, 2H). LC/MS Calcd for [M+H]⁺337.1, 339.1. found 337.0, 339.0.

### Preparation of 3-fluoro-4-[6-methoxy-7-(3-morpholin-4-yl-propoxy)-quinolin-4-yloxy]-phenylamine

A reactor containing 4-(2-fluoro-4-nitro-phenoxy)-6-methoxy-7-(3-morpholin-4-yl propoxy)quinoline (2.5 kg) and 10 percent palladium on carbon (50 percent water wet, 250 g) in a mixture of ethanol and water containing concentrated hydrochloric acid (1.5 L) was pressurized with hydrogen gas (approximately 40 psi). The mixture was stirred at ambient temperature. When the reaction was complete (typically 2 hours), as evidenced by in process HPLC analysis, the hydrogen was vented and the reactor inerted with argon. The reaction mixture was filtered through a bed of Celite® to remove the catalyst. Potassium carbonate was added to the filtrate until the pH of the solution was approximately 10. The resulting suspension was stirred at 20-25° C. for approximately 1 hour. The solids were collected by filtration, washed with water and dried at 50-60° C. under reduced pressure to afford the title compound (1.164 kg). ¹H NMR (400 MHz, DMSO-d6): δ 8.45 (d, 1H), 7.51 (s, 1H), 7.38 (s, 1H), 7.08 (t, 1H), 6.55 (dd, 1H), 6.46 (dd, 1H), 6.39 (dd, 1H), 5.51 (br. s, 2H), 4.19 (t, 2H), 3.94 (s, 3H), 3.59 (t, 4H), 2.47 (t, 2H), 2.39 (m, 4H), 1.98 (m, 2H). LC/MS Calcd for [M+H]⁺428.2. found 428.1.

### Preparation of 1-(4-fluoro-phenylcarbamoyl)-cyclopropanecarboxylic acid

Triethylamine (7.78 kg) was added to a cooled (approximately 4° C.) solution of commercially available cyclopropane 1,1-dicarboxylic acid (9.95 kg) in THF, at a rate such that the batch temperature did not exceed 10° C. The solution was stirred for approximately 30 minutes and then thionyl chloride (9.14 kg) was added, keeping the batch temperature below 10° C. When the addition was complete, a solution of 4 fluoroaniline (9.4 kg) in THF was added at a rate such that the batch temperature did not exceed 10° C. The mixture was stirred for approximately 4 hours and then diluted with isopropyl acetate. The diluted solution was washed sequentially with aqueous sodium hydroxide, water, and aqueous sodium chloride. The organic solution was concentrated by vacuum distillation. Heptane was added to the concentrate. The resulting slurry was filtered by centrifugation and the solids were dried at approximately 35° C. under vacuum to afford the title compound (10.2 kg). ¹H NMR (400 MHz, DMSO-d6): δ 13.06 (br s, 1H), 10.58 (s, 1H), 7.65-7.60 (m, 2H), 7.18-7.12 (m, 2H), 1.41 (s, 4H), LC/MS Calcd for [M+H]⁺ 224.1. found 224.0.

### Preparation of 1-(4-fluoro-phenylcarbamoyl)-cyclopropanecarbonylchloride

Oxalyl chloride (291 mL) was added slowly to a cooled (approximately 5° C.) solution of 1-(4-fluoro-phenylcarbamoyl)-cyclopropanecarboxylic acid in THF at a rate such that the batch temperature did not exceed 10° C. When the addition was complete, the batch was allowed to warm to ambient temperature and held with stirring for approximately 2 hours, at which time in process HPLC analysis indicated the reaction was complete. The solution was used in the next step without further processing.

### Preparation of cyclopropane-1,1-dicarboxylic acid {3-fluoro-4-[6-methoxy-7-(3-morpholin-4-yl-propoxy)-quinolin-4-ylamino]phenyl}-amide-(4 fluorophenyl)-amide

The solution from the previous step was added to a mixture of 3-fluoro-4-[6-methoxy-7-(3-morpholin-4-yl-propoxy)-quinolin-4-yloxy]-phenylamine (1160 kg) and potassium carbonate (412.25 g) in THF and water at a rate such that the batch temperature was maintained at approximately 15-21° C. When the addition was complete, the batch was warmed to ambient temperature and held with stirring for approximately 1 hour, at which time in process HPLC analysis indicated the reaction was complete. Aqueous potassium carbonate solution and isopropyl acetate were added to the batch. The resulting 2-phase mixture was stirred and then the phases were allowed to separate. The aqueous phase was back extracted with isopropyl acetate. The combined isopropyl acetate layers were washed with water followed by aqueous sodium chloride and then slurried with a mixture of magnesium sulfate and activated carbon. The slurry was filtered over Celite® and the filtrate was concentrated to an oil at approximately 30° C. under vacuum to afford the title compound which was carried into the next step without further processing. ¹H NMR (400 MHz, DMSO-d6): δ 10.41 (s, 1H), 10.03 (s, 1H), 8.47 (d, 1H), 7.91 (dd, 1H), 7.65 (m, 2H), 7.53 (m, 2H), 7.42 (m, 2H), 7.16 (t, 2H), 6.41 (d, 1H), 4.20 (t, 2H), 3.95 (s, 3H), 3.59 (t, 4H), 2.47 (t, 2H), 2.39 (m, 4H), 1.98 (m, 2H), 1.47 (m, 4H). LC/MS Calcd for [M+H]⁺633.2. found 633.1.

### Preparation of the bisphosphate salt of cyclopropane-1,1-dicarboxylic acid {3-fluoro-4-[6-methoxy-7-(3-morpholin-4-yl-propoxy)-quinolin-4-ylamino]phenyl}-amide (4-fluoro-phenyl)-amide

Cyclopropane-1,1-dicarboxylic acid {3-fluoro-4-[6-methoxy-7-(3-morpholin-4-yl-propoxy)-quinolin-4-ylamino]phenyl}-amide-(4 fluoro phenyl)-amide from the previous step was dissolved in acetone and water. Phosphoric acid (85%, 372.48 g) was added at a rate such that the batch temperature did not exceed 30° C. The batch was maintained at approximately 15-30° C. with stirring for 1 hour during which time the product precipitated. The solids were collected by filtration, washed with acetone and dried at approximately 60° C. under vacuum to afford the title compound (1.533 kg). The title compound has a c-Met IC₅₀ value of less than 50 nM. The bisphosphate salt is not shown in scheme 1. ¹H NMR (400 MHz, DMSO-d6): (diphosphate) δ 10.41 (s, 1H), 10.02 (s, 1H), 8.48 (d, 1H), 7.93 (dd, 1H), 7.65 (m, 2H), 7.53 (d, 2H), 7.42 (m, 2H), 7.17 (m, 2H), 6.48 (d, 1H), 5.6 (br s, 6H), 4.24 (t, 2H), 3.95 (s, 3H), 3.69 (bs, 4H), 2.73 (bs, 6H), 2.09 (t, 2H), 1.48 (d, 4H).

### Procedure for Direct Coupling

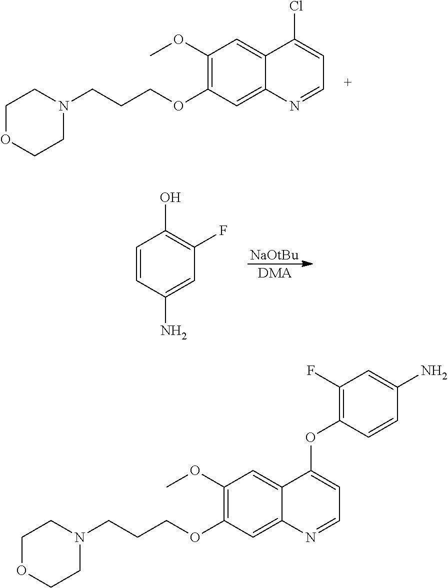

Solid sodium tert-butoxide (1.20 g; 12.5 mmol) was added to a suspension of the chloroquinoline (3.37 g; 10 mmol) in dimethylacetamide (35 mL), followed by solid 2-fluoro-4-hydroxyaniline. The dark green reaction mixture was heated at 95-100° C. for 18 hours. HPLC analysis showed approximately. 18 percent starting material remaining and approximately 79 percent product. The reaction mixture was cooled to below 50° C. and additional sodium tert-butoxide (300 mg; 3.125 mmol) and aniline (300 mg; 2.36 mmol) were added and heating at 95-100° C. was resumed. HPLC analysis after 18 h revealed less than 3% starting material remaining. The reaction was cooled to below 30° C., and ice water (50 mL) was added while maintaining the temperature below 30° C. After stirring for 1 hour at room temperature, the product was collected by filtration, washed with water (2×10 mL) and dried under vacuum on the filter funnel, to yield 4.11 g of the coupled product as a tan solid (96% yield; 89%, corrected for water content). ¹H NMR and MS: consistent with product; 97.8% LCAP; approximately 7 weight percent water by KF.

### Preparation of Compound 2 Hydrate Form

The hydrate of Compound 1 was prepared by adding 4.9614 g of Compound 1 and 50 mL of n-propanol to a 250 mL beaker. The suspension was heated to 90° C. with stirring via a magnetic stir bar at 200 rpm. After 2 hours, the solids were fully dissolved in an amber solution. At the 1 hour and 2 hour time points, 10 mL of n-propanol was added to account for evaporative effects and return the volume of the solution to 50 mL. The solution was then hot-filtered through a 1.6 μm glass fiber filter. The solution was then allowed to dry overnight in the beaker to a powder, which was then redissolved in 150 mL of a 1:1 mixture of acetone and water, and slurried overnight (16 hours) with a foil lid to prevent evaporation. The slurried solids were then collected by vacuum filtration. The final weight recovered was 3.7324 g (75% yield). This batch was stored at ambient conditions for several days prior to analysis.

Karl Fisher water content determinations were performed using a standard procedure. Water content was measured with a Brinkmann KF1V4 Metrohm 756 Coulometer equipped with a 703 Ti stirrer and using Hydranal Coulomat AG reagent. Samples were introduced into the vessel as solids. Approx 30-35 mg of sample was used per titration. A sample of crystalline Compound (I) prepared in Example 1.1.2 was measured in duplicate and was found to have an average water content be 2.5% w/w, with each replicate agreeing to within 0.1%.

A gravimetric vapor sorption (GVS) study was run using a standard procedure. Samples were run on a dynamic vapor sorption analyzer (Surface Measurement Systems) running DVSCFR software. Sample sizes were typically 10 mg. A moisture adsorption desorption isotherm was performed as outlined below. The standard isotherm experiment, performed at 25° C., is a two-cycle run, starting at 40% RH, increasing humidity to 90% RH, decreasing humidity to 0% RH, increasing humidity again to 90% RH, and finally decreasing humidity to 0% RH in 10% RH intervals. The crystalline Compound 2 prepared in Example 1.1.1 showed a 2.5% weight gain at 25° C. and 90% humidity. The GVS sorption and desorption curves showed evidence that the hydrate behaves as an isomorphic desolvate (Stephenson, G. A.; Groleau, E. G.; Kleeman, R. L.; Xu, W.; Rigsbee, D. R. J. Pharm. Sci. 1998, 87, 536-42).

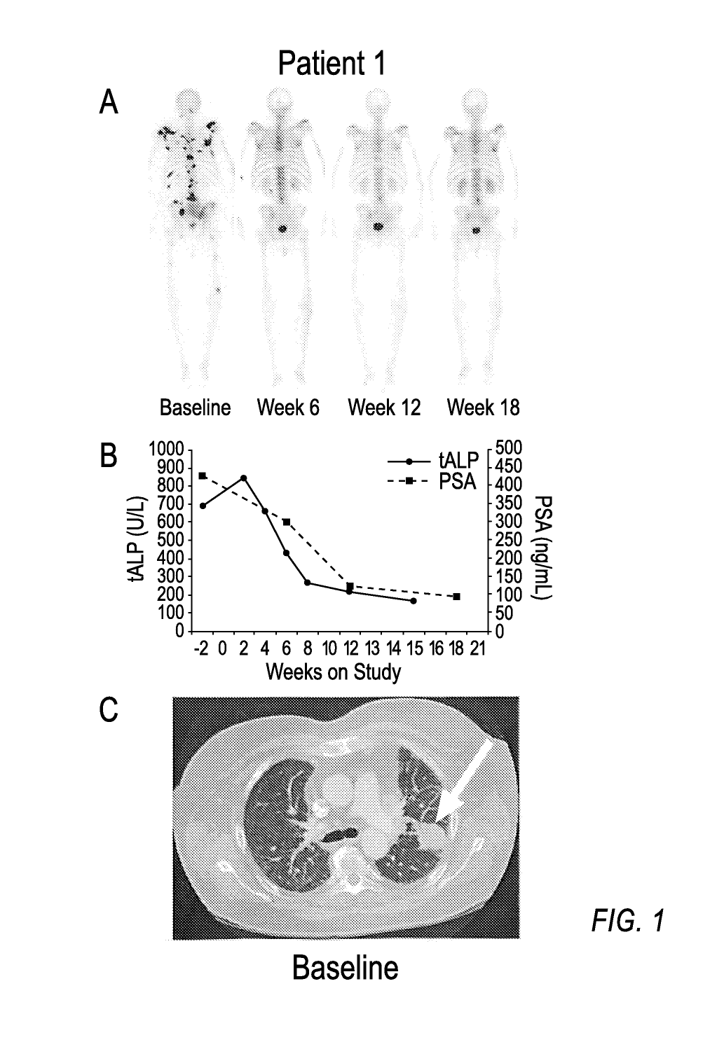

The X-ray powder diffraction pattern of Compound 2 crystalline hydrate prepared above was acquired using a PANalytical X′Pert Pro diffractometer. The sample was gently flattened onto a zero-background silicon insert sample holder. A continuous 20 scan range of 2° to 50° was used with a CuKα radiation source and a generator power of 40 kV and 45 mA. A 2θ step size of 0.017 degrees/step with a step time of 40.7 seconds was used. Samples were rotated at 30 rpm. Experiments were performed at room temperature and at ambient humidity. FIG. 1-B shows the XRPD pattern for N-[3-fluoro-4-({6-(methyloxy)-7-[(3-morpholin-4-ylpropyl)oxy]quinolin-4-yl}oxy)phenyl]-N-(4-fluorophenyl)cyclopropane-1,1-dicarboxamide crystalline hydrate. The following peaks at an experimental °2θ+0.1 °2θ were identified in the XRPD pattern: 6.6, 9.0, 10.2, 12.0, 12.2, 13.1, 13.3, 14.6, 15.6, 16.2, 17.0, 17.1, 17.4, 18.2, 18.4, 18.7, 20.0, 20.3, 20.8, 21.7, 22.1, 23.1, 23.4, 23.8, 24.2, 24.5, 25.0. Only peaks below 25 °2θ are given as these are generally preferred for the identification of crystalline pharmaceutical forms. The entire list of peaks, or a subset thereof, may be sufficient to characterize the hydrate of Compound 2.

DSC thermograms were acquired using a TA Instruments Q2000 differential scanning calorimeter. A sample mass of 2.1500 mg of Compound 2 crystalline hydrate was weighed out directly into an aluminum DSC pan. The pan was sealed by applying pressure by hand and pushing each part the pan together (also known as a loose lid configuration). The temperature was ramped from 25° C. to 225° C. at 10° C./minute. A peak melting temperature of 137.4° C. and a heat flow of 44.2 J/g was measured for the melting endotherm. After the melting event, recrystallization occurs to an anhydrous form, which then melts at 194.1° C.

TGA thermograms were acquired using a TA Instruments Q500 Thermogravimetric Analyzer. The sample pan was tared, and 9.9760 milligrams of Compound (I) crystalline hydrate was placed in the pan. The temperature was ramped from 25° C. to 300° C. at 10° C./minute. A weight loss of 2.97% was observed up to 160° C., with an additional weight loss beyond 200° C. from decomposition.

### Preparation of Compound 2 Crystalline Hydrate with Different Hydration States

Five 150 mg aliquots were taken from the crystalline hydrate batch prepared above and were placed in 10 mL screw-top vials. With the vial tops removed, these aliquots were each stored in chambers with desiccant (Dri-Rite®, tricalcium silicate, RH 2-3%), saturated lithium bromide (6% RH), saturated lithium chloride (11% RH), saturated magnesium chloride (33% RH), and saturated sodium chloride (75% RH). The samples were removed after 2 weeks and immediately sealed with a cap for analysis and characterized.

### Preparation of Compound 3

Compound 3 was prepared as disclosed in WO 2005-030140 as Example 41, pages 206-207 and as disclosed in the following Schemes and Examples.

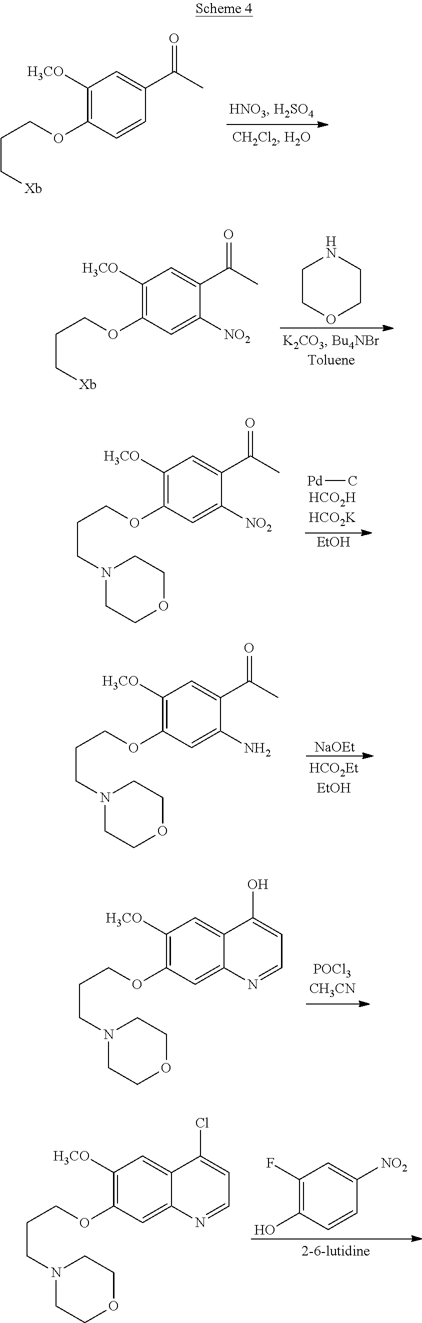

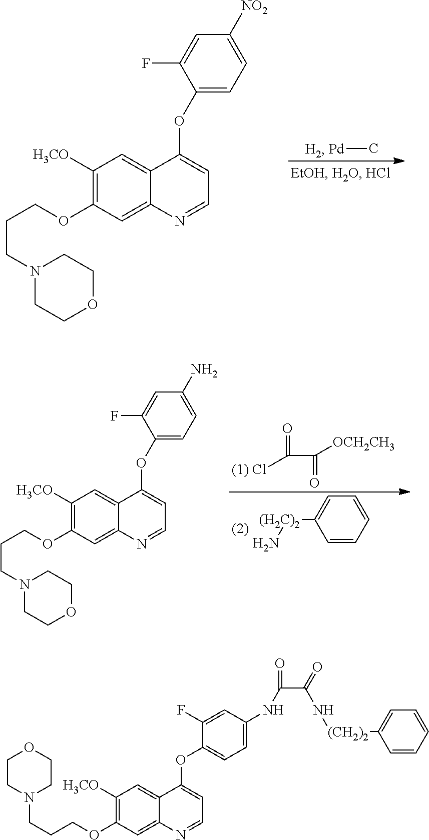

### Preparation of 1-[5 methoxy-4 (3-halo propoxy)-2 nitro-phenyl]-ethanone

A pre-mixed solution of water (80 L) and concentrated sulfuric acid, 96% (88 L) cooled to approximately 5° C. was charged to a reactor containing to the solution of 1-[4-(3-halo propoxy)-3-methoxy phenyl]ethanone (both of which are commercially available) at a rate such that the batch temperature did not exceed approximately 18° C. The resulting solution was cooled to approximately 5° C., and 65% nitric acid (68 L) was added at a rate such that batch temperature did not exceed approximately 10° C. HPLC analysis was used to determine when the reaction was complete. Methylene chloride (175 L) was charged to a separate reactor containing cooled water (1800 L; by dissolving 450 Kg of ice in 1500 of water). The acidic reaction mixture was then added into this mixture. The methylene chloride layer was separated, and the aqueous layer was back extracted with methylene chloride (78 L). The combined methylene chloride layers were washed with two portions of a solution of aqueous sodium bicarbonate followed by water (50 L) and then concentrated by vacuum distillation. 1-Butanol (590 L) was added, and the mixture was again concentrated by vacuum distillation. The resulting solution was stirred at approximately 20° C. during which time the product crystallized. The solids were recovered by filtration, washed with heptane (100 L) to afford the title compound (89.8 kg wet). Mother liquor was concentrated and the resulting solid was filtered and washed with n-heptane (45 L) to afford second crop of the title compound (25 kg wet). Both product crops were combined and dried in a tumble drier at 35° C. to yield product (99.7 kg; 25.6% LOD) which was used directly in the next step without further drying. Three production batches were made. ¹HNMR (400 MHz, DMSO-d6): δ. 7.69 (s, 1H), 7.24 (s, 1H); 4.23 (m, 2H), 3.94 (s, 3H), 3.78 (t)-3.65 (t) (2H), 2.51 (s, 3H), 2.30-2.08 (m, 2H) LC/MS Calcd for [M(Cl)+H]⁺288.1. found 288.0; Calcd for [M(Br)+H]⁺332.0, 334.0. found 331.9, 334.0.

### Preparation of 1-[5-methoxy-4-(3-morpholin-4-yl-propoxy)-2-nitro-phenyl]-ethanone

The solvent wet cake isolated (82.8 kg wet; 74.2 kg dry calc.) in the previous step was dissolved in toluene (390 L). A solution of sodium iodide (29.9 kg) and potassium carbonate (53.4.0 kg) dissolved in water (170 L) was added to this solution, followed by tetrabutylammonium bromide (8.3 kg) and morpholine (67 L). The resulting two-phase mixture was heated to approximately 85° C. for about 10 hours (the reaction completion was tested by an in-process HPLC). The mixture was then cooled to ambient temperature. The organic layer was separated. The aqueous layer was back extracted with toluene (103 L). The combined toluene layers were washed sequentially with two portions of 5% sodium thiosulfate (259 L each) [sodium thiosulfate (26.8 kg) dissolved in water (550 L)] followed by two portions of aqueous NaCl (256 L; NaCl; 15 kg dissolved in water; 300 L). The resulting solution was concentrated under vacuum and n-heptane (340 L) was then charged. The resulting slurry was filtered, washed with n-heptane (75 L) to yield the title compound (92% AUC, HPLC 82.8 wet; 67.2 dry calculated) which was used in the next step without drying. Four manufacturing batches were carried out for this step. ¹HNMR (400 MHz, DMSO-d6): δ. 7.64 (s, 1H), 7.22 (s, 1H), 4.15 (t, 2H), 3.93 (s, 3H), 3.57 (t, 4H), 2.52 (s, 3H), 2.44-2.30 (m, 6H), 1.90 (quin, 2H); LC/MS Calcd for [M+H]⁺ 339.2. found 339.2.

### Preparation of 1-[2-amino-5-methoxy-4-(3-morpholin-4-yl-propoxy)-phenyl]-ethanone

The product from the previous step (30.3 kg) followed by ethanol (22 L) and 10% palladium on carbon (Pd—C; 50% water wet, 2.75 kg) were charged to a reactor The resulting slurry was heated to approximately 48° C., and a solution of formic acid (12 L), potassium formate (22.6 kg), and water (30.8 L) was added. When the addition was complete and the reaction was deemed complete by HPLC, water (130 L) was added to dissolve the byproduct salts. The mixture was filtered to remove the insoluble catalyst. The Pd—C cake was washed with fresh water (25 L). The filtrate was concentrated under reduced pressure, and toluene (105 L) was added. The mixture was made basic (pH=10) by the addition of aqueous potassium carbonate (70 L; K₂CO₃; 28.9 kg dissolved in 115 L of water). Methylene chloride (20 L) was then charged. The organic layer was separated, and sodium chloride (26.3 kg) was charged to the aqueous layer which was back extracted with toluene (125 L). The combined organic phases were washed with potassium carbonate (45 L from above described aqueous potassium carbonate solution) and water (135 L), phases separated. The organic phase was combined with toluene (110 L) and concentrated under vacuum followed by another charge of toluene (110 L) which was again concentrated under vacuum. The drying was confirmed by an in-process testing (Karl Fisher). The resulting solution containing the title compound was used in the next step without further processing. ¹HNMR (400 MHz, DMSO-d6): δ. 7.11 (s, 1H), 7.01 (br s, 2H), 6.31 (s, 1H), 3.97 (t, 2H), 3.69 (s, 3H), 3.57 (t, 4H), 2.42 (s, 3H), 2.44-2.30 (m, 6H), 1.91 (quin, 2H LC/MS Calcd for [M+H]⁺309.2. found 309.1.

### Preparation of 6-methoxy-7-(3-morpholin-4-yl-propoxy)-quinolin-4-ol dihydrochloride dehydrate

A solution of sodium ethoxide (98 L; 21% in ethanol) and ethyl formate (37 L) was added to the solution from the previous step. The solution was warmed to approximately 46° C. for approximately 3 hours. After the reaction was deemed complete by HPLC, water (100 L) was charged to the mixture and the solution was made acidic (pH=1) by the addition of concentrated HCl (37%; 50 L) To the aqueous phase, acetone (335 L) was charged, and the mixture was cooled to approximately 10° C. and stirred for 5 h resulting in a slurry. The product was collected by filtration, and the product was washed with acetone (60 L) and dried under reduced pressure at approximately 40° C. The dried title compound (33.8 kg) was shown by HPLC to be 98% pure (percent area under the curve [AUC] by HPLC). Six lots of the title compound following procedure described were manufactured. ¹HNMR (400 MHz, DMSO-d6): δ. 11.22 (br s, 1H), 8.61 (d, 1H), 7.55 (s, 1H), 7.54 (s, 1H), 7.17 (d, 1H), 4.29 (t, 2H), 3.99 (m, 2H), 3.96 (s, 3H), 3.84 (t, 2H), 3.50 (d, 2H), 3.30 (m, 2H), 3.11 (m, 2H), 2.35 (m, 2H), LC/MS Calcd for [M+H]⁺319.2. found 319.1.

### Preparation of 4-chlor-6-methoxy-7-(3 morpholin-4-yl)-quinoline

Phosphorous oxychloride (59.5 kg) was added to a solution of compound from the previous step (40.0 kg) in acetonitrile (235 L) that was heated to 50-55° C. When the addition was complete, the mixture was heated to reflux (approximately 82° C.) and held at that temperature with stirring for approximately 10 hours, at which time it was sampled for in-process HPLC analysis. The reaction was deemed complete when not more than 5% starting material remained. The reaction mixture was then cooled to 20-25° C. and methylene chloride (100 L) charged. The resulting mixture was then quenched in pre-mixed methylene chloride (155 L), ammonium hydroxide (230 L) and ice (175 kg) while the temperature was maintained below 30° C. The resulting two-phase mixture was separated, and the aqueous layer was back extracted with methylene chloride (110 L). The combined methylene chloride phase was washed with water (185 L) and concentrated under vacuum (to a residual volume 40 L). This was used in the next step without further processing. ¹HNMR (400 MHz, DMSO-d6): δ. 8.61 (d, 1H), 7.56 (d, 1H), 7.45 (s, 1H), 7.38 (s, 1H), 4.21 (t, 2H), 3.97 (s, 3H), 3.58 (m, 2H), 2.50-2.30 (m, 6H), 1.97 (quin, 2H) LC/MS Calcd for [M+H]⁺458.2. found 458.0.

### Preparation of 4-(2-fluoro-4-nitro-phenoxy)-6-methoxy-7-(3-morpholin-4-yl propoxy) quinoline

A solution of the product (from the previous step) and 2-fluoro-4-nitrophenol (16.8 kg) in 2,6-lutidine (55 L) was heated to approximately 160° C., with stirring, for approximately 3 hours, at which time it was sampled for in-process HPLC analysis. The reaction was considered complete with the conversion of compound from the previous step (>83%, HPLC). The reaction mixture was then cooled to approximately 75° C., and water (315 L) was added. Potassium carbonate (47.5 kg) dissolved in water (90 L) was added to the mixture, which was then stirred at ambient temperature overnight. The solids that precipitated were collected by filtration, and then washed with water (82 L). The wet solid was dissolved in methylene chloride (180 L) and aqueous potassium carbonate (65 L, 5%, by weight) charged, stirred for 0.4 h and the phases were separated. This operation was repeated four times and the resulting solution was concentrated under vacuum at 35° C. (residual volume, 40 L). T-butylmethylether (85 L) was then charged and distillation continued under vacuum at 35° C. (residual volume, 50 L). This operation was repeated three times. The wet solid was then heated to approximately 52° C. in MTBE (70 L) for 0.3 h. The solid was filtered, washed with MTBE (28 L). This operation was repeated twice. The wet solid was dried under vacuum at 35-45° C. under reduced pressure to afford 4-(2-fluoro-4-nitro-phenoxy)-6-methoxy-7-(3-morpholin-4-yl-propoxy) quinoline, the title compound (20.2 kg, 99% AUC). Two batches of the title compound were produced. ¹HNMR (400 MHz, DMSO-d6): δ 8.54 (d, 1H), 8.44 (dd, 1H), 8.18 (m, 1H), 7.60 (m, 1H), 7.43 (s, 1H), 7.42 (s, 1H), 6.75 (d, 1H), 4.19 (t, 2H), 3.90 (s, 3H), 3.56 (t, 4H), 2.44 (t, 2H), 2.36 (m, 4H), 1.96 (m, 2H). LC/MS Calcd for [M+H]⁺337.1, 339.1. found 337.0, 339.0.

### Preparation of 3-fluoro-4-[6-methoxy-7-(3-morpholin-4-yl-propoxy)-quinolin-4-yloxy]-phenylamine

A reactor containing the product from the previous step (20.4 kg) and 10% palladium on carbon (50% water wet, 4.3 kg) in a mixture of ethanol (100 L) and water (87 L) containing concentrated hydrochloric acid (12.5 L) was pressurized with hydrogen gas (approximately 5 bar). The temperature of the reaction mixture was not allowed to exceed 46° C. When the reaction was complete, as evidenced by in-process HPLC analysis (typically 2 hours), the hydrogen gas was vented, and the reactor was inerted with nitrogen. The reaction mixture was filtered through a bed of Celite™ to remove the catalyst. Aqueous potassium carbonate (65 L, 5%) was charged to adjust pH (approximately 10). The resulting slurry was filtered washed with water (63 L). The wet solid was suspended in acetonitrile (55 L) and water (55 L), and then the reaction mixture was stirred for approximately 0.3 h. The solid was filtered, washed sequentially with water (35 L), acetonitrile (35 L) and toluene (35 L). The solid was suspended in toluene (100 L) and dried by azeotropic distillation. The Azeotropic step was repeated three times. Finally, the toluene suspension was cooled, and the solids were filtered, washed with toluene (15 L), and dried at 40-45° C. under reduced pressure to afford the title compound (13.9 kg; 100% AUC). Two batches of the title compound were produced. ¹H NMR (400 MHz, DMSO-d6): δ 8.45 (d, 1H), 7.51 (s, 1H), 7.38 (s, 1H), 7.08 (t, 1H), 6.55 (dd, 1H), 6.46 (dd, 1H), 6.39 (dd, 1H), 5.51 (br. s, 2H), 4.19 (t, 2H), 3.94 (s, 3H), 3.59 (t, 4H), 2.47 (t, 2H), 2.39 (m, 4H), 1.98 (m, 2H). LC/MS Calculated for [M+H]⁺ 428.2. found 428.1.

### Procedure for Direct Coupling

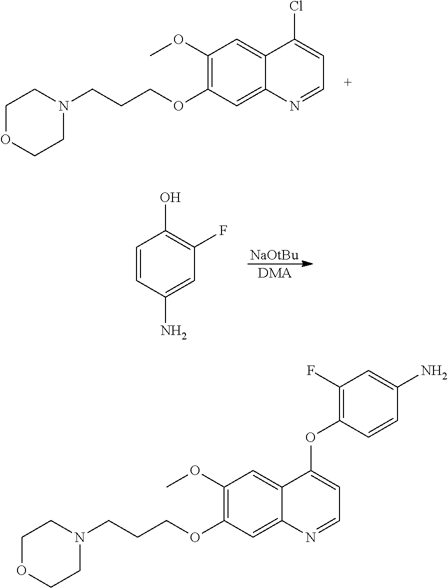

Solid sodium tert-butoxide (1.20 g; 12.5 mmol) was added to a suspension of the chloroquinoline (3.37 g; 10 mmol) in dimethylacetamide (35 mL), followed by solid 2-fluoro-4-hydroxyaniline. The dark green reaction mixture was heated at 95-100° C. for 18 h. HPLC analysis showed ca. 18% starting material remaining and ca. 79% product. The reaction mixture was cooled to below 50° C. and additional sodium tert-butoxide (300 mg; 3.125 mmol) and aniline (300 mg; 2.36 mmol) were added and heating at 95-100° C. was resumed. HPLC analysis after 18 h revealed <3% starting material remaining. The reaction was cooled to below 30° C., and ice water (50 mL) was added while maintaining the temperature below 30° C. After stirring for 1 h at room temperature, the product was collected by filtration, washed with water (2×10 mL) and dried under vacuum on the filter funnel, to yield 4.11 g of the coupled product as a tan solid (96% yield; 89%, corrected for water content). ¹H NMR and MS: consistent with product; 97.8% LCAP; ˜7 wt % water by KF.

### Preparation of N-{3-Fluoro-4-[6-methoxy-7-(3-morpholin-4-yl-propoxy)-quinolin-4-yloxy]-phenyl}-N′-phenethyl-oxalamide

Compound from the previous step (13.7 kg), dimethyl formamide (70 L), and triethylamine (6.8 kg) were charged to a reactor. The reactor contents were cooled to approximately 5° C., and ethyl chlorooxoacetate (5.2 kg) was added so that the reaction temperature was maintained below 25° C. After the reaction was complete (typically 2-4 hours; determined by HPLC when <2% AUC compound from the previous step remained), a solution of 2-phenylethylamine (10.0 kg) in tetrahydrofuran (40 L) was charged to the reactor while maintaining the reaction temperature below 30° C. The reaction was deemed complete (typically complete in 2-4 hours) when <2% AUC ethyl ester remained by HPLC. The reactor contents were cooled to 20-25° C., and charged to a mixture of ice (44 kg), water (98 L) and ethanol (144 L) at a rate to maintain the temperature below 20° C. This was followed by stirring the reactor contents for at least 5 hours at 20-25° C.; the resulting slurry was concentrated under vacuum at 50° C. Water was then charged and the resulting solid precipitate that was recovered by filtration, washed with a mixture of ethanol (100 L) and water (100 L), and dried under vacuum at 60-65° C. to afford the title compound (16.9 kg; 98.7%, HPLC) which was used in the next step.

A second batch of this step was produced employing a similar methodology but resulted in lesser title compound. This was subjected to re-crystallization using the following strategy:

The title compound (17.2 kg) was suspended in THF (172 L), heated to approximately 60° C. and water, and was charged until complete dissolution was achieved. Ethanol (258 L) was then added and the mixture was cooled to approximately 25° C. and stirred for at least 8 h. The resulting slurry was filtered; and the solid was washed with a mixture of ethanol/water (1:1, 168 L). The product was dried under vacuum at approximately 50° C. to yield title compound (10.1 kg; 98.3%, HPLC). ¹H NMR (400 MHz, CDCl₃): [symbol] 9.37 (s, 1H), 8.46 (d, 1H), 7.81 (dd, 1H), 7.57 (t, 1H), 7.53 (s, 1H), 7.42 (s, 2H), 7.34-7.20 (m, 6H), 6.39 (d, 1H), 4.27 (t, 2H), 4.03 (s, 3H), 3.71 (m, 4H), 3.65 (q, 2H), 2.91 (t, 2H), 2.56 (br s, 4H), 2.13 (m, 2H); ¹³C NMR (100 MHz, d₅-DMSO): 5160.1, 160.0, 159.5, 155.2, 152.7, 152.6, 150.2, 149.5, 147.1, 139.7, 137.3, 137.1, 129.3, 129.1, 126.9, 124.8, 117.9, 115.1, 109.2, 102.7, 99.6, 67.4, 66.9, 56.5, 55.5, 54.1, 41.3, 35.2, 26.4; IR (cm⁻¹): 1655, 1506, 1483, 1431, 1350, 1302, 1248, 1221, 1176, 1119, 864, 843, 804, 741, 700; LC/MS Calcd for (M+H): 603.66. found 603.

### Preparation of N-{3-Fluoro-4-[6-methoxy-7-(3-morpholin-4-yl-propoxy)-quinolin-4-yloxy]-phenyl}-N′-phenethyl-oxalamide bis phosphate

The compound from the previous step (16.8 kg) was charged to a reactor, and ethanol (170 L) was added. Phosphoric acid (10%, 72.6 kg) was added at a rate such that the batch temperature did not exceed 30° C. The batch was then heated to approximately 60° C. with stirring for 3 hours to ensure total dissolution. The batch was then cooled to 20-25° C. and stirred for approximately 6 hours during which time the product precipitated. The solids were collected by filtration, washed twice with ethanol (152 L), and dried at 55-60° C. under vacuum to afford title compound (18.0 kg). A second batch of the title compound (9.9 kg) using similar strategy was produced. ¹H NMR (400 MHz, DMSO-d6): δ 11.04 (s, 1H), 9.14 (t, 1H), 8.48 (d, 1H), 8.04 (dd, 1H), 7.84 (br d, 1H), 7.55 (s, 1H), 7.50 (t, 1H), 7.46 (br s, 1H), 7.32 (m, 2H), 7.24 (m, 3H), 6.48 (d, 1H), 4.24 (br s, 2H), 3.96 (s, 3H), 3.74 (bs, 4H), 3.48 (q, 2H), 2.85 (m, 8H), 2.14 (br s, 2H).

### Case Studies

The MET and VEGF signaling pathways appear to play important roles in osteoblast and osteoclast function. Strong immunohistochemical staining of MET has been observed in both cell types in developing bone. HGF and MET are expressed by osteoblasts and osteoclasts in vitro and mediate cellular responses such as proliferation, migration, and expression of ALP. Secretion of HGF by osteoblasts has been proposed as a key factor in osteoblast/osteoclast coupling, and in the development of bone metastases by tumor cells that express MET. Osteoblasts and osteoclasts also express VEGF and its receptors, and VEGF signaling in these cells is involved in potential autocrine and/or paracrine feedback mechanisms regulating cell migration, differentiation, and survival.

Bone metastases are present in 90% of patients with castration-resistant prostate cancer (CRPC), causing significant morbidity and mortality. Activation of the MET and VEGFR signaling pathways is implicated in the development of bone metastases in CRPC. Three metastatic CRPC patients treated with Compound 1, an inhibitor of MET and VEGFR, had dramatic responses with near complete resolution of bone lesions, marked reduction in bone pain and total serum alkaline phosphatase (tALP) levels, and reduction in measurable disease. These results indicate that dual modulation of the MET and VEGFR signaling pathways is a useful therapeutic approach for treating CRPC.

Compound 1 is an orally bioavailable multitargeted tyrosine kinase inhibitor with potent activity against MET and VEGFR. Compound 1 suppresses MET and VEGFR signaling, rapidly induces apoptosis of endothelial cells and tumor cells, and causes tumor regression in xenograft tumor models. Compound 1 also significantly reduces tumor invasiveness and metastasis and substantially improves overall survival in a murine pancreatic neuroendocrine tumor model. In a phase 1 clinical study, Compound 1 was generally well-tolerated at a 100 mg dose, with fatigue, diarrhea, anorexia, rash, and palmar-plantar erythrodysesthesia being the most commonly observed adverse events.

Compound 2 is an orally bioavailable multitargeted tyrosine kinase inhibitor with potent activity against MET and VEGFR. Compound 2 suppresses MET and VEGFR signaling, rapidly induces apoptosis of endothelial cells and tumor cells, and causes tumor regression in xenograft tumor models. Compound 2 also significantly reduces tumor invasiveness and metastasis and substantially improves overall survival in a murine pancreatic neuroendocrine tumor model. In clinical studies, Compound 2 was administered at up to a 240 mg dose.

Based on target rationale and observed antitumor activity in clinical studies, an adaptive phase 2 trial was undertaken in multiple indications including CRPC (ClinicalTrials.gov: NCT00940225), in which Compound 1 was administered as a 100 mg dose to patients. The findings in the first three CRPC patients with evidence of bone metastases on bone scan enrolled to this study are described in the following Case Studies.

Baseline characteristics for patients 1-3 are summarized in Table 1.

TABLE 1
Summary of Baseline Characteristics and Preliminary Best Responses for CRPC
Patients Treated with Compound 1.

| | Patient 1 | Patient 2 | Patient 3 |
|---|---|---|---|
| Baseline Characteristics | | | |
| Age (years) | 77 | 73 | 66 |
| Diagnosis | 1993 | 2009 | 2009 |
| ECOG performance status | 1 | 0 | 1 |
| Disease location(s) | Lung, LN, bone | Liver, LN, bone | LN, bone |
| Prior cancer therapies | Radical | Radiation to | CAB, docetaxel |
| | prostatectomy, | pubic ramus and | |
| | radiation to | acetabulum, | |
| | prostate bed, | CAB | |
| | CAB, DES, | | |
| | docetaxel | | |
| Bisphophonates | No | No | Yes |
| Narcotics | Yes | No | No |
| Pain | Yes | Yes | Yes |
| PSA (ng/mL) | 430.4 | 14.7 | 2.8 |
| tALP (U/L) | 689 | 108 | 869 |
| Hemoglobin (g/dL) | 13.5 | 13.3 | 10.2 |
| Summary of Best Responses | | | |
| Tumor response | −41% | −20% | −51% |
| Bone scan | Complete | Improvement | Near resolution |
| | resolution | | |
| Pain | Improvement | Pain-free | Pain-free |
| PSA | −78% | +61% | −57% |
| tALP | −77% | −6% | −77% |
| Hemoglobin (g/dL) | +1.4 | +1.8 | +2.2 |

ADT, androgen-deprivation therapy;
CAB, combined androgen blockade (leuprolide + bicalutamide);
DES, diethylstilbestrol;
LN, lymph node;
PSA, prostate-specific antigen;
tALP, total alkaline phosphatase.

Patient 1 was diagnosed with localized prostate cancer in 1993 and treated with radical prostatectomy (Gleason score unavailable; PSA, 0.99 ng/mL). In 2000, local disease recurrence was treated with radiation therapy. In 2001, combined androgen blockade (CAB) with leuprolide and bicalutamide was initiated for rising PSA (3.5 ng/mL). In 2006, diethystillbestrol (DES) was administered briefly. In 2007, 6 cycles of docetaxel were given for new lung metastases. Rising PSA was unresponsive to antiandrogen withdrawal. Androgen ablation therapy was continued until clinical progression. In October 2009, bone metastasis to the spine associated with impingement on the spinal cord and back pain, was treated with radiation therapy (37.5 Gy). In February 2010, a bone scan was performed due to increasing bone pain and showed diffuse uptake of radiotracer in the axial and appendicular skeleton. A CT scan revealed new pulmonary and mediastinal lymph node metastases. PSA was 430.4 ng/mL.

Patient 2 was diagnosed in April of 2009 after presenting with a pathologic fracture (Gleason score, 4+5=9; PSA, 45.34 ng/mL). Bone scan showed uptake of radiotracer in the left iliac wing, left sacroiliac joint, femoral head, and the pubic symphysis. Biopsy of the left pubic ramus confirmed metastatic adenocarcinoma with mixed lytic and blastic lesions. CAB with leuprolide and bicalutamide and radiation therapy (8 Gy) to the left pubic ramus and acetabulum resulted in bone pain relief and PSA normalization. Rising PSA in November 2009 (16 ng/mL) was unresponsive to antiandrogen withdrawal. In February 2010, bone scan showed multiple foci throughout the axial and appendicular skeleton. A CT scan revealed retroperitoneal lymph node enlargement and liver metastases (PSA, 28.1 ng/mL). Further progression of disease was marked by recurrent bone pain, new lung and hepatic metastases.

Patient 3 was diagnosed in April 2009 after presenting with right hip pain (Gleason score, 4+5=9; PSA, 2.6 ng/mL). Bone scan showed uptake of radiotracer at multiple sites throughout the axial and appendicular skeleton. A CT scan revealed retroperitoneal, common iliac, and supraclavicular adenopathy. CAB with leuprolide and bicalutamide was initiated. The patient received 6 cycles of docetaxel through December 2009. Following treatment, a bone scan showed no changes. A CT scan revealed near resolution of the retroperitoneal and common iliac adenopathy. In March 2010, PSA began to rise, and bone pain worsened. A repeat bone scan showed new foci, and a CT scan showed an increase in the retroperitoneal, para-aortic, and bilateral common iliac adenopathy. Rising PSA in April 2010 (2.8 ng/mL) and increasing bone pain were unresponsive to antiandrogen withdrawal.

### Results

All patients provided informed consent before study screening.

Patient 1 started Compound 1 on Feb. 12, 2010. Four weeks later, significant reduction in bone pain was reported. At Week 6, bone scan showed a dramatic decrease in radiotracer uptake by bone metastases (FIG. 1A). A CT scan showed a partial response (PR) with a 33% decrease in measurable target lesions (FIG. 1C). At Week 12, near complete resolution of bone lesions and a 44% decrease in target lesions was observed and was stable through Week 18. Corresponding with the bone scan response, after an initial rise, serum tALP levels decreased from 689 U/L at baseline to 159 U/L at Week 18 (FIG. 1B and Table 1). In addition, there was an increase in hemoglobin of 1.4 g/dL at Week 2 compared with baseline (Table 1). PSA decreased from 430 ng/mL at baseline to 93.5 ng/mL at Week 18 (FIG. 1B and Table 1). The patient was on open-label treatment through Week 18 when he withdrew after developing Grade 3 diarrhea.

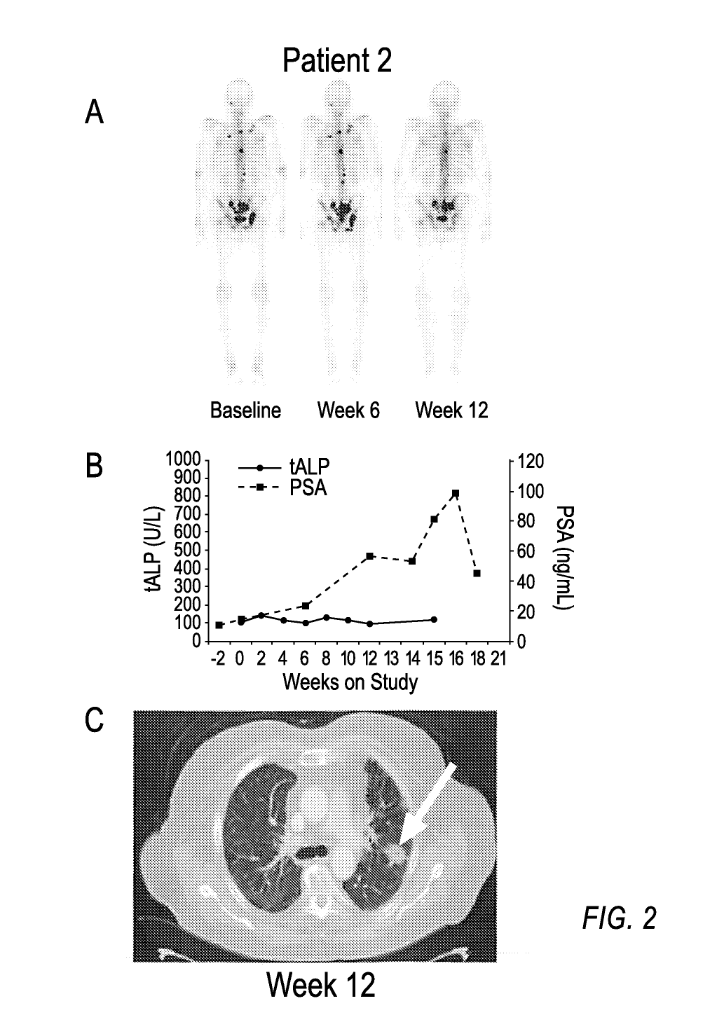

Patient 2 started Compound I on Mar. 31, 2010. At Week 4, reduction in bone pain was reported. At Week 6, bone scan showed a slight flair in radiotracer uptake by bone lesions (FIG. 2A), and a CT scan showed a 13% decrease in target lesions (FIG. 2C). At Week 12, a substantial reduction of radiotracer uptake (FIG. 2A) and a 20% decrease in measurable disease were observed (Table 1). After randomization to placebo at Week 12 the patient developed severe bone pain and sacral nerve root impingement. Radiation to the spine was administered, and the patient crossed over to open-label Compound 1 treatment at Week 15. Serum tALP levels were within the normal range (101-144 U/L) (FIG. 2B). Hemoglobin increased by 1.8 g/dL at Week 12 compared with baseline (Table 1). PSA peaked at close to 6-fold of baseline by Week 16, but then decreased to 2-fold of baseline by Week 18 subsequent to crossing over to Compound 1 from placebo (FIG. 2B and Table 1). The patient continues on Compound I treatment as of September 2010.

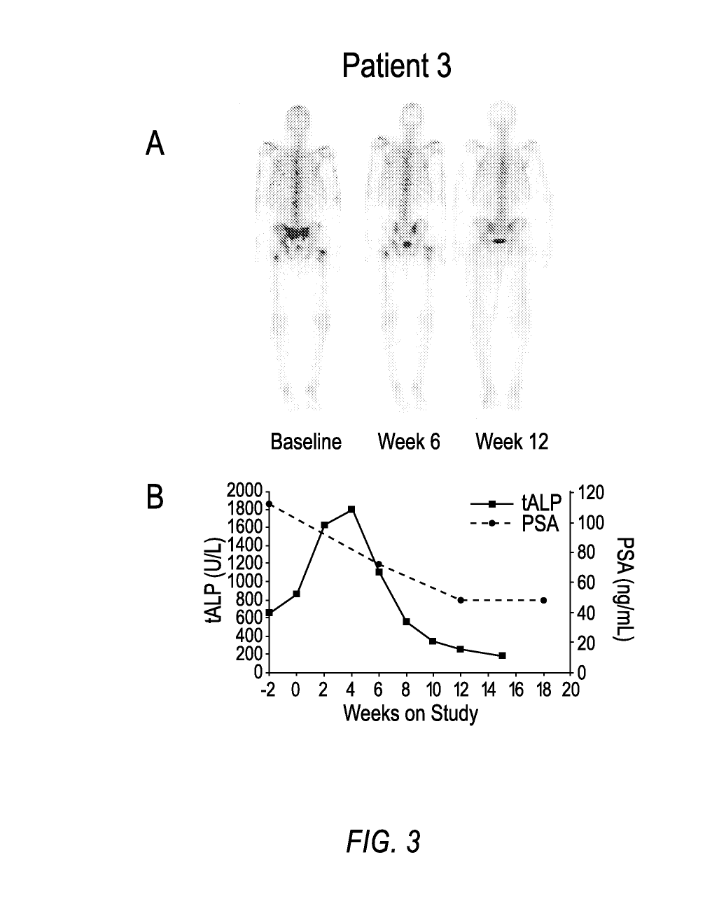

Patient 3 started Compound 1 on Apr. 26, 2010. After three weeks a complete resolution of pain was reported. At Week 6, bone scan showed a dramatic reduction in radiotracer uptake (FIG. 3A), and a CT scan showed a PR with a 43% decrease in measurable target lesions. At Week 12 a complete resolution of bone lesions on bone scan (FIG. 3A) and a 51% decrease in measurable disease was observed (Table 1 and FIG. 3B)). After an initial rise, serum tALP levels steadily decreased, with tALP at 869 U/L at baseline and 197 U/L at Week 18 (FIG. 3B and Table 1). Hemoglobin increased 2.2 g/dL at Week 2 compared with baseline (Table 1). PSA decreased from 2.4 ng/mL at screening to 1.2 ng/mL at Week 18 (FIG. 3B and Table 1). The patient continues on Compound 1 treatment as of September 2010.

### Discussion

All three patients experienced a striking decrease in uptake of radiotracer on bone scan upon treatment with Compound 1. These findings were accompanied by substantial reductions in bone pain and evidence of response or stabilization in soft tissue lesions during therapy with Compound I. The onset of the effect was very rapid in two of the patients, with substantial improvement or near resolution of bone scan and improvement in pain occurring in the first 6 weeks. In the third patient, an apparent flare in the bone scan was observed at 6 weeks, followed by improvement by 12 weeks. To our knowledge, such a comprehensive and rapid impact on both osseous and soft tissue disease has not been observed in this patient population.

Uptake of radiotracer in bone depends on both local blood flow and osteoblastic activity, both of which may be pathologically modulated by the tumor cells associated with the bone lesion. Resolving uptake may therefore be attributable to either interruption of local blood flow, direct modulation of osteoblastic activity, a direct effect on the tumor cells in bone, or a combination of these processes. However, decreased uptake on bone scan in men with CRPC has only been rarely noted with VEGFNEGFR targeted therapy, despite numerous trials with such agents. Similarly, observations of decreased uptake on bone scan in CRPC patients have only been reported rarely for abiraterone, which targets the cancer cells directly, and for dasatinib, which targets both cancer cells and osteoclasts. Thus, targeting angiogenesis alone, or selectively targeting the tumor cells and/or osteoclasts, has not resulted in effects similar to those observed in the patients treated with Compound I.

The results reported here indicate a potential critical role for the MET and VEGF signaling pathways in the progression of CRPC and point to the promise that simultaneously targeting these pathways may have in reducing morbidity and mortality in this patient population

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1-18. (canceled)

19. A method for treating osteoblastic bone metastases, castration resistant prostate cancer (CRPC), or bone cancer associated with prostate cancer comprising administering a compound that dually modulates MET and VEGF to a patient in need of such treatment a compound of Formula II: 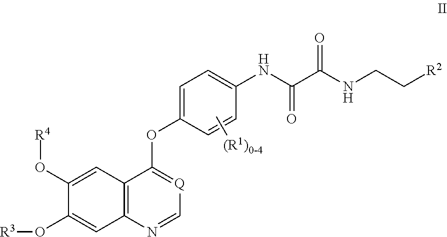
or a pharmaceutically acceptable salt thereof, wherein:
R¹ is halo;
R² is optionally substituted phenyl;
R³ is (C₁-C₆)alkyl substituted with heterocycloalkyl;
R⁴ is (C₁-C₆)alkyl; and
Q is CH or N.

20. The method of claim 1, wherein the compound of Formula II is Compound 3:

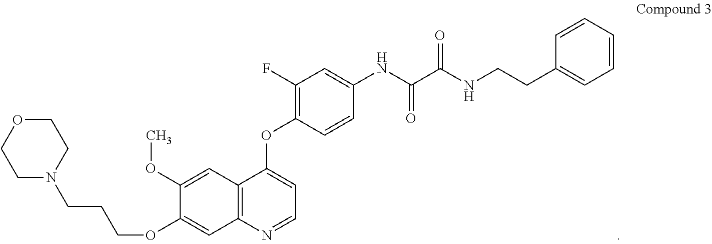 [SMILES: COc1cc2c(Oc3ccc(NC(=O)C(=O)NCCc4ccccc4)cc3F)ccnc2cc1OCCCN1CCOCC1]

21. The method of claim 2, wherein the disease is osteoblastic bone metastases, or osteoblastic bone metastases associated with prostate cancer.

22. The method of claim 2, wherein the disease is CRPC, or bone cancer associated with CRPC.

23. The method of claim 2, wherein the disease is osteoblastic bone metastases associated with CRPC.

24. A method for ameliorating abnormal deposition of unstructured bone accompanied by increased skeletal fractures, spinal cord compression, and severe bone pain of osteoblastic bone metastases compound of Formula II or pharmaceutically acceptable salt thereof, as defined in claim 1 or claim 2, for use in ameliorating abnormal deposition of unstructured bone accompanied by increased skeletal fractures, spinal cord compression, and severe bone pain of osteoblastic bone metastases.

25. A pharmaceutical composition comprising a compound of Formula II or pharmaceutically acceptable salt thereof, as defined in claim 2, and a pharmaceutically acceptable carrier, excipient, or diluent.
